(19) United States
(12) Patent Application Publication
(10) Pub. No.: US 2024/0296919 A1
(43) Pub. Date: Sep. 5, 2024

(54) LEARNING A SEQUENTIAL DIFFUSION MODEL FOR THE FORWARD AND INVERSE PROBLEM IN SIMULATION OF PHYSICAL SYSTEMS

(71) Applicant: NEC Laboratories Europe GmbH, Heidelberg (DE)

(72) Inventors: Francesco Alesiani, Heidelberg (DE); Makoto Takamoto, Heidelberg (DE); Henrik Christiansen, Heidelberg (DE)

(21) Appl. No.: 18/350,831

(22) Filed: Jul. 12, 2023

Related U.S. Application Data
(60) Provisional application No. 63/442,142, filed on Jan. 31, 2023.

(51) Int. Cl. G16C 10/00

(52) U.S. Cl. CPC G16C 20/70

(57) ABSTRACT

A method for simulating physical systems using a sequential diffusion model (SDM) comprising a denoising model includes collecting training data for training the SDM. The method further includes training the denoising model using the training data such that the SDM models a forward and/or reverse problem for a simulation of a physical system over a period of time, and generating a solution for the physical system based on training the denoising model. The solution indicates a final condition of the physical system at a final instance in the period of time for the forward problem and an initial condition of the physical system at an initial instance in the period of time for the reverse problem.

### CROSS-REFERENCE TO PRIOR APPLICATION

Priority is claimed to U.S. Provisional Application No. 63/442,142, filed on Jan. 31, 2023, the entire contents of which is hereby incorporated by reference herein.

### FIELD

The present invention relates to a method, system and computer-readable medium for learning a sequential diffusion model, or a recurrent denoising diffusion model, for the forward and/or inverse problem in the simulation of physical systems, such as for molecular dynamics, drug or material discovery, or for other machine learning tasks, such as those that use or learn partial differential equations.

### BACKGROUND

When developing new medication (e.g., drugs) or more generally evaluating properties of material at the molecular level, there is a need to simulate the physical evolution of the system, that is, investigate how the system evolved over a period of time. This can also apply when modeling fluids such as fluids found in aerodynamic applications or when modeling wave equations in seismic applications. Starting from an initial condition, the system can evolve over a period of time. The inverse process of investigation can derive the initial condition from observations, which can be used to analyze the soil for example for geological exploration or determine the center of an earthquake. This means that it is necessary to simulate the physical system in the forward time (e.g., when the initial condition(s)) are known and/or solve the inverse problem (e.g., when only the final solution is observed).

Current methods model these processes either by using numerical simulation, where all the equations are known in advance, or training surrogate models. These current models are limited in accuracy of the prediction, especially when multiple initial conditions can generate the same final solution or when the physical process is governed by an unknown stochastic process, such as in systems governed by stochastic differential equations (e.g., where the energy distribution of a molecular system is modeled at various temperatures).

### SUMMARY

In an embodiment, the present disclosure provides a method for simulating physical systems using a sequential diffusion model (SDM) and the SDM comprises a denoising model. Training data for training the SDM is collected. The denoising model is trained using the training data such that the SDM models a forward and/or reverse problem for a simulation of a physical system over a period of time. A solution is generated for the physical system based on training the denoising model. The solution indicates a final condition of the physical system at a final instance in the period of time for the forward problem and an initial condition of the physical system at an initial instance in the period of time for the reverse problem.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 illustrates an example use of a sequential diffusion model according to an embodiment of the present invention practically applied for protein binding for drug development (training and test phases);

FIG. 2 illustrates surrogate models which are trained separately and then used to simulate new scenarios (active learning) according to an embodiment of the present invention;

FIG. 3 illustrates the training process of the sequential diffusion model for the forward problem according to an embodiment of the present invention;

FIG. 4 illustrates the generation process of the sequential diffusion model for the forward problem according to an embodiment of the present invention;

FIG. 5 illustrates the training of the sequential diffusion model for the inverse problem according to an embodiment of the present invention;

FIG. 6 illustrates the generation of the sequential diffusion model for the inverse problem according to an embodiment of the present invention;

FIG. 7 illustrates the forward model (on the left) and backward model (on the right) according to an embodiment of the present invention;

FIG. 8 illustrates the training sequence for a sequential diffusion model according to an embodiment of the present invention;

FIG. 9 illustrates the training sequence for a sequential diffusion model according to another embodiment of the present invention;

FIG. 10 illustrates a diagram for the inverse problem with linear transformation according to an embodiment of the present invention;

FIG. 11 illustrates a method for performing diffusion and denoising in a latent space according to an embodiment of the present invention;

FIG. 12 illustrates a method with a two-step implementation of denoising and forward prediction according to an embodiment of the present invention;

FIG. 13 is a visualization of the two-step implementation according to an embodiment of the present invention;

FIG. 14 illustrates a method with a one-step implementation and a visualization of the one-step implementation according to an embodiment of the present invention, and shows multiple implementations that consider: one step forward, one forward and one at the same time; one at the same and two forward, or three steps forward;

FIG. 15 illustrates multiple configurations of a forward denoising network according to an embodiment of the present invention;

FIG. 16 illustrates a forward denoising neural network according to an embodiment of the present invention;

FIG. 17 illustrates a temporal attention and temporal encoding for infinite memory and look-ahead according to an embodiment of the present invention;

FIGS. 18A-18C illustrate details of the implementation of the time attention, and additional encoding that is used for the noise, the noise encoding (NE) and the time encoding (TE) according to an embodiment of the present invention;

FIGS. 19A-19C illustrate an efficient denoising process (forward and sliding) according to an embodiment of the present invention;

FIG. 20 illustrates a physical loss to train the network to follow the physical law during the training of the denoising network according to an embodiment of the present invention, where ϵ is some error;

FIG. 21 is a block diagram of an exemplary processing system, which can be configured to perform any and all operations disclosed herein;

FIG. 22 illustrates exemplary code for condition sequential DM training according to an embodiment of the present invention; and

FIG. 23 illustrates exemplary code for sampling of the conditional sequential DM according to an embodiment of the present invention.

### DETAILED DESCRIPTION

Molecular dynamics, and more generally the simulation of a physical system, often requires modeling partial or ordinary differential equations that evolve as a function of time and finds application in technical application domains such as drug design, material design, molecule generation from fragments, absorbent modeling, molecular modeling, and/or property prediction. In an embodiment, the present invention provides a method that can be trained on either simulated or experimental data, allowing to evolve the system both in the forward and backward directions of time.

For example, when developing new drugs or more generally evaluating the properties of a material at a molecular level, the physical evolution of the system at hand is simulated to investigate how the system evolved over time. This also, for example, applies when modeling a fluid in an aerodynamic application or when modeling a wave equation in seismic applications. Starting from an initial condition, the system is evolved. The inverse process of investigation derives the initial condition from the observations, which is used to, for example, analyze the soil for example for geological exploration or determine the center of an earthquake. Accordingly, the physical system can be simulates in the forward time (e.g., where the initial condition is known) or the inverse problem can be solved (e.g., where only the final solution is observed).

In an embodiment, the present invention provides a sequential diffusion model having technical applications in molecular dynamics (MD) and genetic sequencing, and in other technical domains where partial differential equations (PDEs) are used in or solved by a machine learning task. Although a recently proposed diffusion model has shown high quality generative power, it cannot be directly applied to the forward and inverse problem. In an embodiment, the present invention provides a different, improved model, which is also referred to herein as a sequential diffusion model (SDM).

According to a first aspect, the present disclosure provides a method for simulating physical systems using a sequential diffusion model (SDM) comprising a denoising model. The method includes collecting training data for training the SDM and training the denoising model using the training data such that the SDM models a forward and/or reverse problem for a simulation of a physical system over a period of time. The method further includes generating a solution for the physical system based on training the denoising model. The solution indicates a final condition of the physical system at a final instance in the period of time for the forward problem and an initial condition of the physical system at an initial instance in the period of time for the reverse problem.

According to a second aspect, the method according to the first aspect further comprises that collecting the training data comprises generating simulated data and generating the simulated data comprises iteratively requesting a numerical simulator to generate new data based on performance of the SDM in an active learning cycle.

According to a third aspect, the method according to the first or the second aspect further comprises that the physical system is associated with molecule generation, the training data comprises molecular data in a simplified molecular-input line-entry (SMILE) format, the molecular data indicates molecules as proteins, configurations of the molecules, and atom types of the molecules, and the configurations of the molecules are represented in 2-D coordinates or are in 3-D space.

According to a fourth aspect, the method according to any of the first through third aspects further comprises that training the denoising model using the training data comprises training the denoising model based on initial conditions of the physical system, boundary conditions of the physical system, observations or final conditions of the physical system, and past or future time steps of the physical system.

According to a fifth aspect, the method according to any of the first through fourth aspects further comprises that training the denoising model comprises sequentially and recursively updating the SDM to predict noise or a clean input, after adding Gaussian noise to inputs of the SDM and training the denoising model to reconstruct the Gaussian noise.

According to a sixth aspect, the method according to any of the first to fifth aspects further comprises that training the denoising model comprises starting with a noisy version of an input and denoising conditional to the input using the denoising model, and a first portion of the input is fixed and not modified and a new component of the input is generated at each diffusion step of the SDM

According to a seventh aspect, the method according to any of the first to sixth aspects further comprises that training the denoising model further comprises: at each of the diffusion steps of the SDM, generating a denoised sequence by propagating a sequential process either in a forward direction or backward direction. Further, each propagation step obtains conditioning from a previous time step, conditioning on the input, and one or more variables to optimize upon. Also, the input is an initial condition or an observation and the one or more variables indicate boundary conditions or the initial condition.

According to an eighth aspect, the method according to any of the first to seventh aspects further comprises that a condition of the SDM is on a forward model or a reverse model, the forward model and the reverse model are modeled with neural networks, and a boundary condition of the SDM is a discrete variable.

According to an ninth aspect, the method according to any of the first through eighth aspects further comprising that the physical system is a molecular system, a condition of the SDM is an integrator based on a gradient of potential energy, and the gradient of the potential energy is modeled using a neural network.

According to an tenth aspect, the method according to any of the first to ninth aspects further comprises that physical constraints and physical laws are used as a loss function to minimize a denoised prediction of the SDM such that the SDM is physically consistent.

According to an eleventh aspect, the method according to any of the first to tenth aspects further comprises that the physical system is generating a video, and the physical constraints and physical laws indicate a language model and description of the video in words or sentences and/or textual description for consecutive frames of the video changes according to an externally provided distance.

According to an twelfth aspect, the method according to any of the first to eleventh aspects further comprises that generating the solution for the physical system comprises generating a sequence of molecule configurations, generating the sequence of molecule configurations comprises inputting, into the SDM, descriptions of molecules in a simplified molecular-input line-entry (SMILE) format that are converted into a 3-D format and desired properties of a generated output to determine an output, and the output is a 3-D description of a generated molecule in the SMILE format and indicates expected properties of the generated molecule.

According to a thirteenth aspect, the method according to any of the first to twelfth aspects further comprises that generating the solution for the physical system comprises generating a solution of a partial derivative equation (PDE) or a video, the SDM is conditioned to one or more conditions, and the one or more conditions indicate past or future solutions, external input, observations, initial conditions, final conditions, or boundary conditions.

A fourteenth aspect of the present disclosure provides a system for simulating physical systems using a sequential diffusion model (SDM) comprising a denoising model. The system comprises one or more hardware processors, which, alone or in combination, are configured to provide for execution of the following steps: collecting training data for training the SDM; training the denoising model using the training data such that the SDM models a forward and/or reverse problem for a simulation of a physical system over a period of time; and generating a solution for the physical system based on training the denoising model, wherein the solution indicates a final condition of the physical system at a final instance in the period of time for the forward problem and an initial condition of the physical system at an initial instance in the period of time for the reverse problem.

A fifteenth aspect of the present disclosure provides a tangible, non-transitory computer-readable medium having instructions thereon, which, upon being executed by one or more processors, provides for execution of the method according to any of the first to the thirteenth aspects.

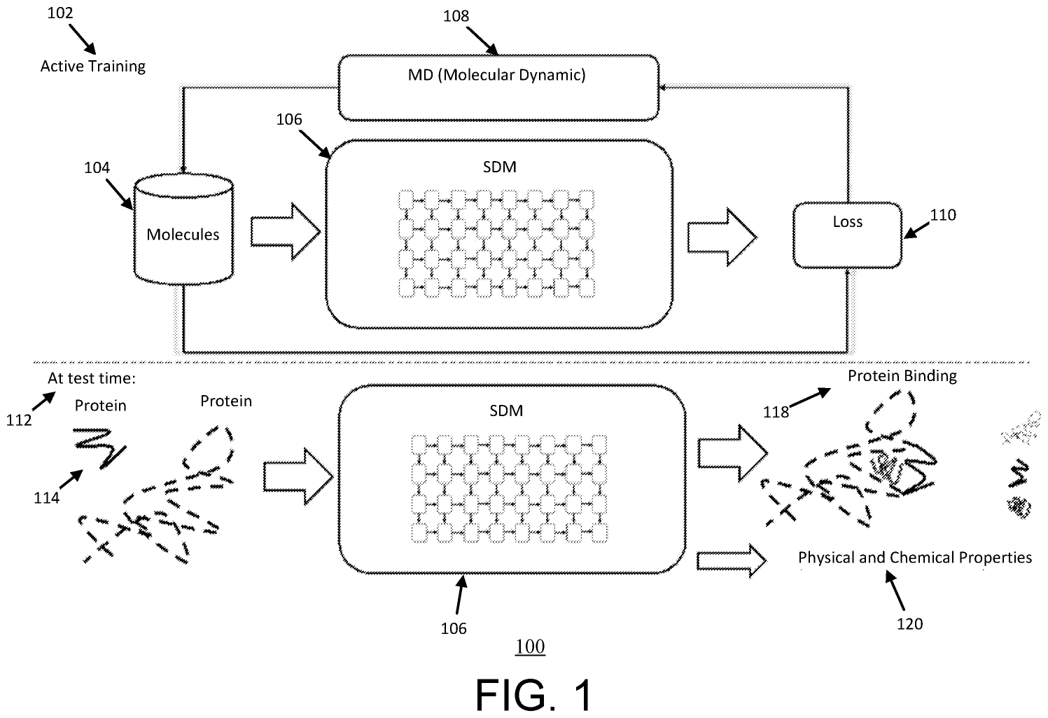

FIG. 1 illustrates an example use of a sequential diffusion model according to an embodiment of the present invention practically applied for protein binding for drug development (training and test phases). For example, FIG. 1 shows an environment 100 for using the SDM for training and then simulating the protein binding process of drug discovery. The fragments of the proteins are set as input and the system produces the molecular dynamic simulation of the final configuration.

For instance, the active training phase 102 includes inputting molecules 104 (e.g., fragments of the proteins) as an input into the SDM 106. A loss 110 (e.g., a loss function) is applied to an output of the SDM 106 and is returned to the molecular dynamic (MD) 108. MDs can be a computer simulation method for analyzing the physical movements of atoms and/or molecules (e.g., the atoms and/or molecules are allowed to interact for a fixed period of time, giving a view of the dynamic evolution of the system). The MD 108 then provides information to the molecules 104 and the training process is repeated.

At test time 112 and/or in actual implementation, the fragments of the proteins 114 are into the SDM 106. The SDM 106 outputs the protein binding 118 and the physical and chemical properties 120. For instance, using a traditional diffusion model, the diffusion model is typically one-dimensional (e.g., noise is iteratively added into and/or removed from an image). However, traditional diffusion models are unable to and do not take into account actual time that has elapsed into a system (e.g., within a time period of a minute or an hour). For instance, the diffusion time (e.g., time of diffusion) of traditional diffusion models is not the same as the time of the physical system (e.g., the temporal time). As such, the use of traditional diffusion models are limited when compared to using the SDM 106. For instance, in some embodiments, the SDM 106 is two-dimensional (described and shown below) and takes into account both the diffusion/de-noising as well as the temporal direction so as to be able to better simulate the physical evolution of the system (e.g., the binding of proteins) over a period of time. In other embodiments, the SDM 106 can be modeled as one-dimensional, three-dimensional, or a higher dimensional model (e.g., for the binding of proteins, the dimensions can represent its probability and stability).

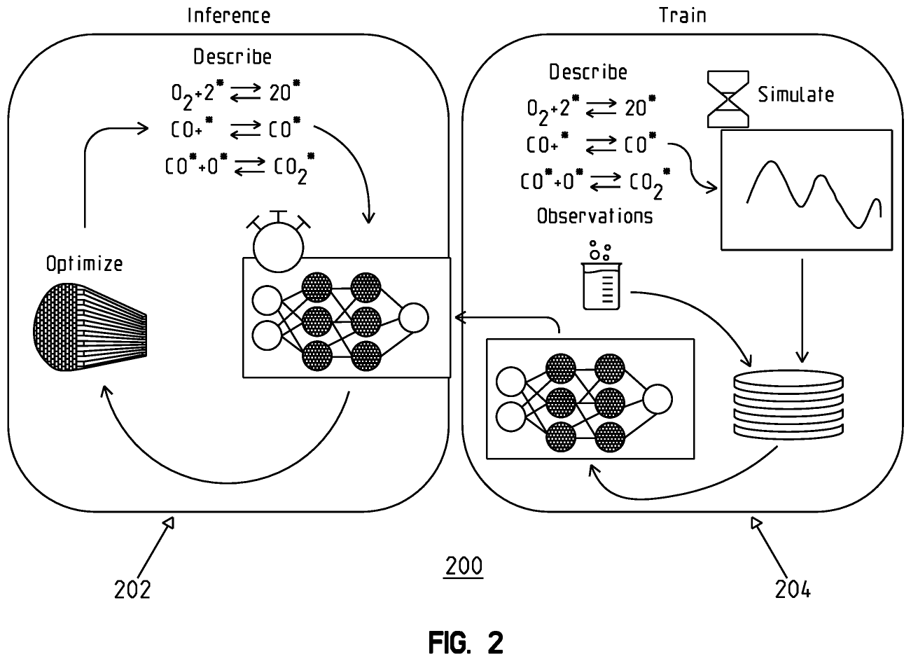

For instance, during training (e.g., active training 102), data is collected using either real data or an expensive molecular dynamic simulator. The data can also be generated in active learning on demand, and the training stops when the performance reaches the expected level of accuracy. The data being generated in active learning on demand is shown in FIG. 2. FIG. 2 illustrates surrogate models which are trained separately and then used to simulate new scenarios (active learning) according to an embodiment of the present invention. For instance, FIG. 2 shows surrogate models 200 that describe the active learning or active training 102 of FIG. 1 in more detail.

For example, FIG. 2 includes an inference portion 202 and a training portion 204. The training portion 204 includes a describe block, which is then used for simulation, and an observation block. The simulations and observations are compiled into data and provided to a model (e.g., the SDM 106), and this is fed into the inference portion 202. The inference portion includes a describe block, the SDM 106, and the optimize block.

Embodiments of the present invention aim to advantageously exploit a denoising diffusion model for solving forward or inverse problems in physical simulation systems (e.g., in a physical environment over a period of time), such as for molecular simulation problems. To achieve this objective, embodiments of the present invention model the underlying process as a sequence in forward (or reverse) time. For instance, in the forward process, embodiments of the present invention start from the initial condition and model the underlying system as the system evolves over a period of time. In the inverse process, embodiments of the present invention derives the initial conditions from the end result. For example, based on the observations (e.g., the damage or other features of an earthquake), a reverse sequence in time is modeled so as to determine the initial conditions (e.g., the initial time when the earthquake occurred and/or the epicenter of the earthquake).

As part of the problem formulation, a sequence of variables x₀, . . . , x_{T} is considered. The sequential diffusion model is used to generate this sequence by estimating the probability density (p(x₀, . . . , x_{T})) from the data. While it may be possible to create an explicit model for the forward or reverse generation procedure, this has some drawbacks similar to drawbacks of the existing technology, in particular:
1. Vanishing gradients and an increase in error with the length of T.
2. Only generating the initial state, and all the sequence would be determined since the evolution is deterministic.

Embodiments of the present invention provide an alternative approach that does not suffer from these and other technical drawbacks. According to an embodiment, a method for learning an SDM is provided, which is also referred to herein as an alternatively recurrent denoising diffusion model, and which provides for learning a forward and inverse recurrent denoising diffusion model, e.g., for modeling partial differential equations (PDEs), molecular dynamic and/or deoxyribonucleic acid (DNA)/ribonucleic acid (RNA) sequences.

According to an embodiment, the present invention provides a special form of a conditional denoising diffusion model, where sampling occurs from a conditional diffusion model over the previous (and/or future) time step. This model can be practically applied to learn: 1) a neural operator for solving PDEs; 2) the inverse problem for PDEs; 3) the forward molecular dynamics problem (from a configuration to generate a stable configuration); 4) a diffusion model to generate a stable configuration from fragments of molecules; 5) a diffusion model to model sequences, as DNA/RNA/mRNA sequences; and/or 6) a diffusion model for video restoration.

In contrast, an alternative approach that naively uses a diffusion model to generate the full sequence would be more computationally complex and would have increased computational cost because it would require a neural network that has the complete sequence as input and output, thus also limiting its use to a fixed length.

Jascha Sohl-Dickstein, et al., “Deep Unsupervised Learning using Nonequilibrium Thermodynamics,” arXiv:1503.03585 (Nov. 18, 2015), which is hereby incorporated by reference herein, describe diffusion models that learn a data distribution p(x) from samples by iteratively denoising a normal distributed random variable, modeled as the reverse (generative) process of a Markov Chain of length K+1 (e.g., the length of the Markov chain is a value, K, plus one). The training is performed by minimizing a variational lower bound on the forward process p(x^{K})Π_{k=K-1}⁰p(x^{k-1}|x^{k}), where p(x^{K}) is the probability of the state x^{k} at the final step K, and p(x^{k-1}|x^{k}) is the conditional probability of the state x^{k-1} at time k−1 given the state x^{k} at time k. These models are implemented as a sequence of denoising autoencoder ∈_{θ}(x^{k}, t); k=0, . . . , K that are trained to a denoised version of the perturbed input variable x^{k}, with a loss function:

$$\\ {ℒ}^{{}_{} DM} = {𝔼}_{x , ϵ \sim 𝒩 ( 0 , 1 ) , k \sim U [ 0 , K ]} [ {‖ ϵ - {ϵ}_{θ} ( {x}^{{}_{} k} , k ) ‖}_{2}^{2} ]$$

where k∈[K] is uniformly sampled. ∈_{θ}(x^{k}, t) is a neural network with input the current state (or latent state) xk and the diffusion time t. ∈˜ [symbol] (0,1) is a random noise with Gaussian distribution of zero mean and unit variance, k=0, . . . , K is the time sequence, k˜U[0, K] the uniform distribution over the interval [0,K], while [symbol] _{x,∈˜} [symbol] _{(0,1),k˜U[0,K]} is the statistical expectation operator on the variables x, ∈, k.

A latent diffusion model (LDM) is considered with latent variables z^{k}. Latent variables can be variables that cannot be observed. The loss function is:

$$\\ {ℒ}^{{}_{} LDM} = {𝔼}_{{ε}_{ϕ} ( x ) , ϵ \sim 𝒩 ( 0 , 1 ) , k} [ {‖ ϵ - {ϵ}_{θ} ( {z}^{{}_{} k} , k ) ‖}_{2}^{2} ]$$

with ε_{ϕ}(x) the trainable encoding function and [symbol] _{ϕ}(z) the decoder network.

A conditional latent diffusion model (CLDM) can be provided as follows:

$$\\ {ℒ}^{{}_{} CLDM} = {𝔼}_{{ε}_{ϕ} ( x ) , ϵ \sim 𝒩 ( 0 , 1 ) , k} [ {‖ ϵ - {ϵ}_{θ} ( {z}^{{}_{} k} , k , {τ}_{ψ} ( y ) ) ‖}_{2}^{2} ]$$

where τ_{ψ}(y) is a neural network with parameters ψ, that represents the encoding of the conditioning variable y. The CLDM is a regular diffusion model, where the (latent) state x^{k} (or the network itself, epsilon, ε) is augmented with an additional external/contextual information (e.g., y), as for example, in image generation, the text describing the image (e.g., “a cat and a dog”). The information is typically embedded in a vector that is processed by an additional network, in this case tau (e.g., τ).

A conditional latent neural network considers a U-Net, a convolutional network architecture, and a Fourier neural operator (FNO) with a cross-attention mechanism (see, e.g., Ashish Vaswani, et al., “Attention is all you need,” arXiv:1706.03762 (Dec. 6, 2017, which is hereby incorporated by reference herein). The cross-attention layer of the

$$\\ U ‐ Net attention ( Q , K , V ) = softmax ( \frac{1}{√ ( d )} {QK}^{{}_{} T} ) V ,$$

$$\\ Q = {W}_{Q}^{{}_{} ( l )} {z}^{{}_{} ( l ) k} , K = {W}_{K}^{{}_{} ( l )} {τ}_{ψ} ( y ) , V = {W}_{V}^{{}_{} ( l )} {τ}_{ψ} ( y )$$

and similar for the FNO, with the attention used to model the latent variable z^{(l+1)k}=attention(Q, K, V)(z^{(l)k}).

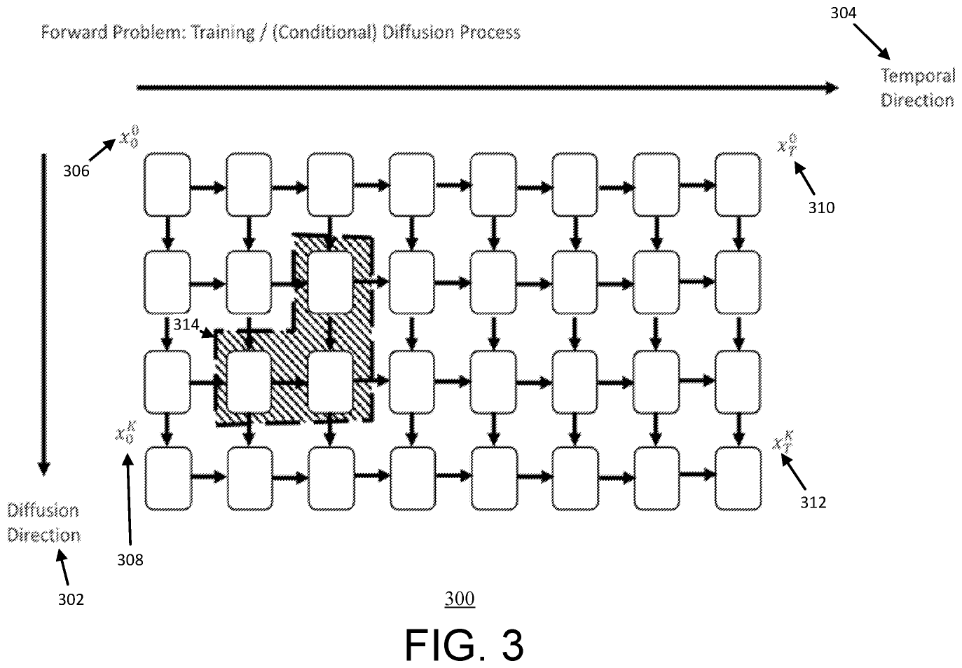

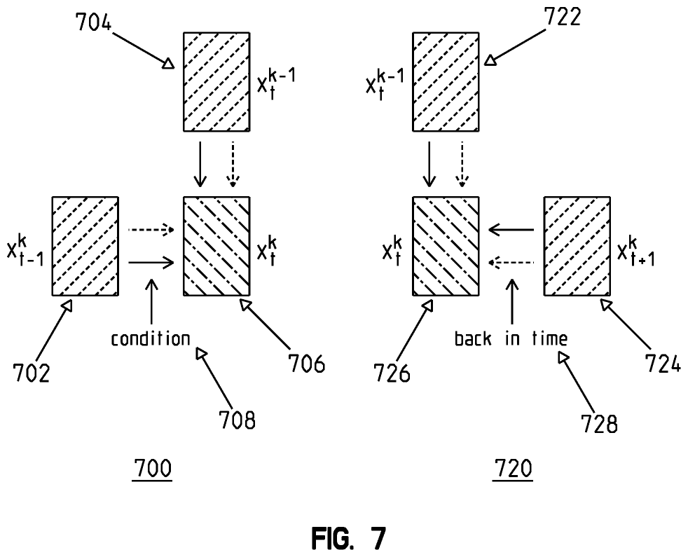

In an embodiment, the present invention provides a method for learning a sequential diffusion model. FIGS. 3-6 illustrate training and generation procedures for a sequential diffusion model for the forward and inverse problems, and FIG. 7 shows the forward (left) and backward (right) conditional models and how the information from the previous (or successive) time step, is used during the diffusion/denoising process.

For instance, FIG. 3 illustrates the training process 300 of the sequential diffusion model for the forward problem according to an embodiment of the present invention. The training process 300 is for the forward problem and training/(conditional) diffusion process. For example, as mentioned above, the forward problem includes understanding the initial condition and then modeling the system (e.g., protein binding) over a period of time. As shown, the initial condition x₀⁰ 306 can move in two-dimensions—the diffusion direction 302 and the temporal direction 304. For instance, traditionally, in the forward problem for the diffusion model, noise is added in steps. Thus, in the top left box of FIG. 3, when noise (e.g., Gaussian noise) is added, the initial condition 306 transitions to a second state such as the block shown immediately below the initial condition block. When additional noise is added, this transitions into a third state (e.g., the third block in the first column), and so on until it reaches an end state for the noise addition (e.g., the condition x₀^{K} 308. As mentioned above, K represents a value associated with the Markov Chain for the diffusion model.

In addition, for the SDM of the present invention and for modeling certain systems that evolve or change over a time span such as protein binding, merely adding noise to the system might not be sufficient enough by itself to understanding the modeling of the entire system as the system evolves over time (e.g., the interaction of the molecules/proteins during the binding process). To account for this, the SDM includes states in the temporal direction 304, and connections (e.g., conditions) for transferring between the states in both the diffusion direction (e.g., the addition of noise) as well as states in the temporal direction (e.g., at a subsequent time interval, period, or instance). For instance, the initial condition 306 can transition both in the y-direction (e.g., the diffusion direction 302) and the x-direction (e.g., the temporal direction), and each subsequent condition can transition in both directions as well. At the end of the temporal direction (e.g., the condition at the end of the first row) is a final temporal condition x_{T}⁰ 310, and the final condition (e.g., the bottom right condition) is an end condition x_{T}^{K} 312. For example, by including the transitions for both the diffusion direction 302 and the temporal direction 304, the SDM models a system's evolution and transition both for the diffusion direction 302 (e.g., from 0 to K) and from the temporal direction 304 (e.g., from 0 to T). The shaded portion 314 shows three conditions three states involved in the training process. The left block represents the conditional information, the upper block represents the block to be denoised, and the lower/right block is the predicted (denoised) block.

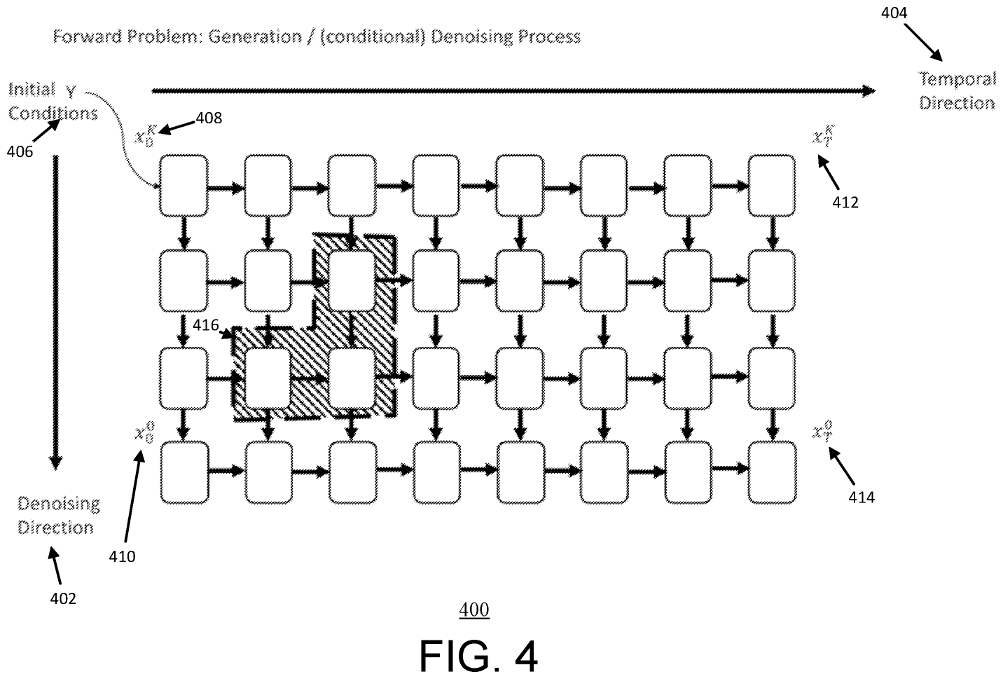

FIG. 4 illustrates the generation process 400 of the sequential diffusion model for the forward problem according to an embodiment of the present invention. For instance, the generation process 400 is for the forward problem and the generation/(conditional) denoising process. For instance, the SDM starts with the initial conditions Y 406, which is provided to the first state x₀^{K} 408. The generation process 400 is similar to the training process 300 except that the diffusion direction 302 is replaced with the denoising direction 402. For instance, instead of including more noise when progressing along the diffusion direction 302, the generation process 400 removes noise when progressing along the denoising direction 402. Therefore, at each transition, along the x-axis, the temporal direction changes (e.g., a subsequent time interval, period, or instance) until the condition (e.g., state) reaches the final temporal condition x_{T}^{K} 412. Along the y-axis, the denoising direction 402 changes at each state to remove noise until the condition reaches the original state x₀⁰ 410, which is similar to the initial condition x₀⁰ 306 of FIG. 3. Similarly, the final condition x_{T}⁰ 414 is at the end of the temporal direction 404 and the denoising direction 402, which is similar to the final temporal condition x_{T}⁰ 310. The shaded portion 416 shows the three blocks involved during the generation process. The left block is the conditional input, the upper is the noisy input, and the lower/right is the denoised block.

Thus, referring to FIGS. 3 and 4, the training process 300 and the generation process 400 show the forward problem (e.g., a problem progressing forwardly in time) using the SDM. For instance, similar to diffusion models, the SDM takes into account the noise to both provide noise in the diffusion direction 302 during training and remove noise in the denoising direction 402 in the generation process. Further, the SDM takes into account the evolution of the system over time in both the generation and training processes 300, 400 by including states/conditions that progress forwardly in the temporal directions 302 and 402. For example, in some embodiment such as in protein binding, positive and negative examples are trained where the diffusion either attracts or does not attract the protein fragment. Starting from the dynamic of the proteins' fragments, the model (e.g., the sequential diffusion model) is trained to reproduce the observed (data) trajectory. During training, scheduled noise is added to the current and previous steps, while the network is used to predict the noise on the current time step. During generation (e.g., inference), embodiments of the present invention start for an arbitrary configuration and generate the trajectory (or multiple trajectories). From these trajectories, embodiments of the present invention can measure the property of the protein, such as the binding probability, the forces involved, and/or the energy.

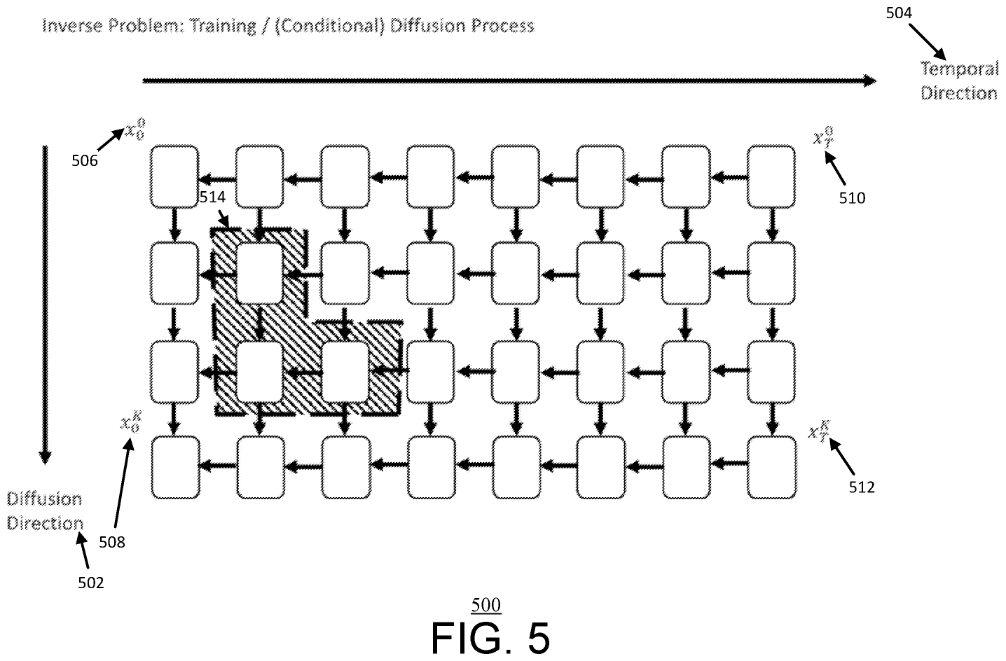

FIG. 5 illustrates the training of the sequential diffusion model for the inverse problem according to an embodiment of the present invention. The training process 500 is for the inverse problem and training/(conditional) diffusion process. For example, FIG. 5 is similar to FIG. 3 with respect to the diffusion direction 502 and the temporal direction 504. However, instead of starting at the initial condition or state x₀⁰ 506 and state x₀^{K} 508, the training process 500 is reversed in the temporal direction 504 (e.g., starting from the end time instance or period, and moving backwards to the beginning of the time instance or period). For instance, the start can occur at state x_{T}⁰ 510 and move in the diffusion direction 502 (e.g., adding noise) to reach state x_{T}^{K} 512 and backwards in time along the temporal direction 504. The shaded portion 514 shows the blocks involved during training of the inverse problem. The block on the right is the conditional input for the denoising network, while the block on the upper part is the input and contains a noisy version for the system at the k time step. The block at the bottom/left is the output block. In training, the network predicts the input error.

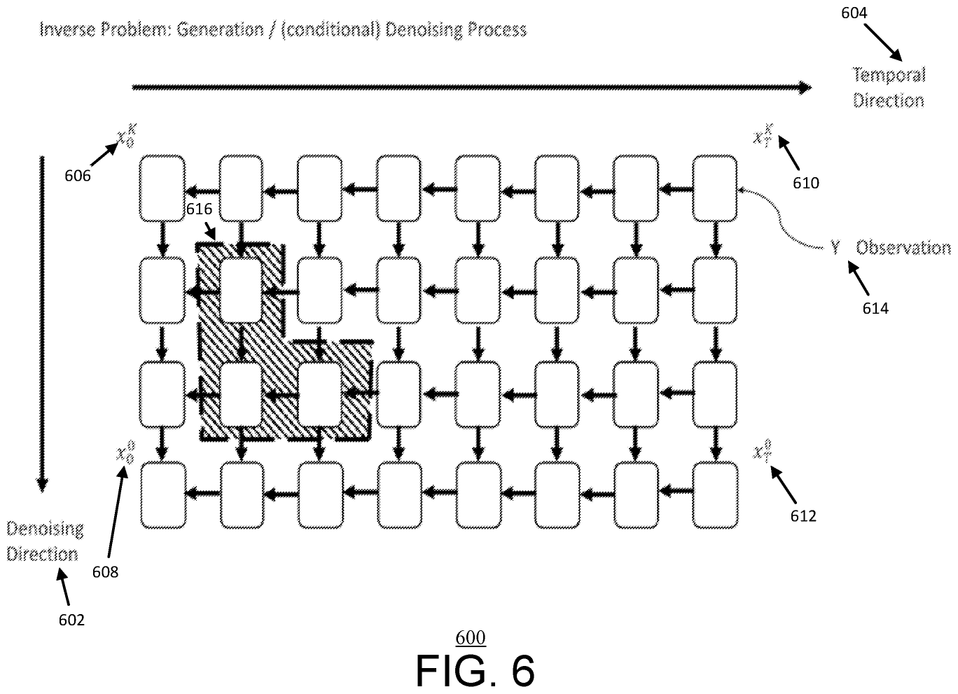

FIG. 6 illustrates the generation of the sequential diffusion model for the inverse problem according to an embodiment of the present invention. For instance, the generation process 600 is for the inverse problem and the generation/(conditional) denoising process. For instance, generation process 600 is similar to generation process 400 except it is for the inverse problem (e.g., moving backwards in time or in the temporal direction 604). The denoising direction 602 and the temporal direction 604 are shown along with an observation Y 614. The states 606, 608, 610, and 612 are shown as well. The shaded portion 616 represents the blocks involved during generation. The right block is the conditional input from the precious step, the block on the top is the input for the denoising network, and the block in the bottom/left is the generated output.

Thus, referring to FIGS. 5 and 6, the training process 500 and the generation process 600 show the inverse problem (e.g., a problem progressing backwards in time) using the SDM. For instance, similar to diffusion models, the SDM takes into account the noise to both provide noise in the diffusion direction 502 during training and remove noise in the denoising direction 602 in the generation process. Further, the SDM takes into account the backwards evolution of the system over time in both the generation and training processes 500, 600 by including states/conditions that progress backwards in the temporal directions 502 and 602. For example, in some embodiments, to model the wave equation of an earthquake, embodiments of the present invention measure the value in the feature, and SDM is used to reverse the process. During the training, embodiments of the present invention generate the forward process for various conditions, and then train the denoting network to remove the scheduled error conditioned to the future time step state. Once the network is trained, embodiments of the present invention can generate multiple trajectories to model the wave propagation of the earthquake.

FIG. 7 illustrates the forward model (on the left) and backward model (on the right) according to an embodiment of the present invention. For example, for the forward model 700, the state xₜ₋₁^{k} 702 and the state xₜ^{k-1} 702 transition to the state xₜ^{k} 706. For instance, by adding or removing noise, the state 704 can transition to the state 706. By applying a condition 708, the state 702 can transition to state 706.

For the backward model 720, the state xₜ^{k-1} 722 and the state xₜ₋₁^{k} 724 transition to the state xₜ^{k} 726. For instance, by adding or removing noise, the state 722 can transition to the state 726. By applying back in time features 728, the state 724 can transition to state 726.

For example, these blocks 702-706 and 722-726 represent the input/output of the denoising network. The architecture (input/output) changes based on the direction of the temporal propagation. The “condition” 708 and “back in time” 728 refer to the arrows showing the transition from blocks 702 to 706 and blocks 724 to 726. They are not used to describe additional input even if additional input that for example, represents some additional contextual information (e.g., the temperature or the presence of external factors), can be included. In some instances, the blocks represent the state of the system. For example, in some embodiments, the state 702 (e.g., block 702) is the molecule at the previous diffusion step, state 704 is the state at the previous time-step, and state 706 is the predicted state based on the noise level in 702. In FIG. 7, the blocks are shown differently than in FIGS. 3-6. For instance, the time dimension (k) is vertical while the diffusion direction is horizontal (t) in FIG. 7.

In an embodiment, a partial differential equation (PDE) can then be considered in the form of:

$$\\ {δ}_{t} u ( x , t ) = {δ}_{x} u ( x , t ) + …$$

where δₜ, δₓ are the partial derivatives with respect to time (t) and space (x), while u(x, t) is the solution of the PDE. The neural operator is F:u(x, t−1)→u(x, t), thereby providing the sequence:

$$\\ u ( x , t ) = F ( u ( x , t - 1 ) , B )$$

where u(x, 0) is the initial state and B represents the boundary conditions (e.g., constraints necessary for the solution of a boundary value problem). It is now possible to determine the CLDM for the exemplary PDE equation and its inverse.

Forward CLDM (F-CLDM): In order to generate the sequence at time t using the diffusion process, the state variable (xₜ^{k}) and conditional variable (yₜ^{k}) are set to:

$$\\ {x}_{t}^{{}_{} k} = {u}^{{}_{} k} ( x , t ) , {y}_{t}^{{}_{} k} = {u}^{{}_{} k} ( x , t - 1 )$$

For instance, the above (e.g., the F-CLDM) describes FIGS. 3 and 4 in equations for the PDE case. For example, the latent variable represents the state x with a latent variable obtained with two additional networks that map the input to the latent space and back from the latent space to the original space.

An alternative conditioning function is a forward function ƒ_{θ}, which can be trained separately on the input sequence (thus giving a prior information to improve convergence) as follows:

$$\\ {x}_{t}^{{}_{} k} = {u}^{{}_{} k} ( x , t ) , {y}_{t}^{{}_{} k} = {f}_{θ} ( {u}^{{}_{} k} ( x , t - 1 ) )$$

Inverse CLDM (I-CLDM): Similarly, the inverse problem can be defined, where it is desired to generate the sequence for a time t using the diffusion process from t+1, as follows:

$$\\ {x}_{t}^{{}_{} k} = {u}^{{}_{} k} ( x , t ) , {y}_{t}^{{}_{} k} = {u}^{{}_{} k} ( x , t + 1 )$$

For instance, the above (e.g., the Inverse CLDM) describes the inverse problem, and these equations describe the inverse architecture in FIGS. 5-6. Also, in this case, the latent space is modeled with two additional neural networks.

An alternative conditioning function is a backward function ƒ_{θ}, as follows:

$$\\ {x}_{t}^{{}_{} k} = {u}^{{}_{} k} ( x , t ) , {y}_{t}^{{}_{} k} = {g}_{θ} ( {u}^{{}_{} k} ( x , t + 1 ) )$$

where g_{θ}(u(x, t)) is the inverse propagation function.

As an alternative conditioning mechanism, the conditioning can happen also with a feature-wise linear modulation (FiLM) layer or the Taylor/channel conditioning. For instance, regarding FIGS. 3-7, there might not be any visual differences between this alternative conditioning mechanism and the above. But, in some embodiments, FiLM represents an alternative approach to include the conditional information and is less expensive in terms of parameters.

An embodiment of the present invention models discrete variables as follows: 1) propagating the gradient using straight through; 2) using implicit maximum likelihood estimation (iMLE, i.e. perturb and MAP, see Pasquale Minervini, et al., “Adaptive Perturbation-Based Gradient Estimation for Discrete Latent Variables,” arXivL2209.04862 (Sep. 11, 2022), which is hereby incorporated by reference herein); 3) using one hot-encoding and then using the argmax; and/or 4) using a Markov chain in the discrete variable. For instance, discrete variables can be used to model categorical information as, for example, the atom type or the presence/absence of space or presence of obstacle to represent the boundary conditions. The training can change since there is computation of either the derivative of discrete variables or use of some encoding of the variable as Markov chains and train the transition probability matrix.

Thus, the forward sequential diffusion model and inverse sequential diffusion model become:

$$\\ {x}_{t}^{{}_{} k} = {u}^{{}_{} k} ( x , t ) , {y}_{t}^{{}_{} k} = {f}_{θ} ( {u}^{{}_{} k} ( x , t - 1 ) , B )$$

$$\\ {x}_{t}^{{}_{} k} = {u}^{{}_{} k} ( x , t ) , {y}_{t}^{{}_{} k} = {g}_{θ} ( {u}^{{}_{} k} ( x , t + 1 ) , B )$$

where the variables are concatenated.

In an embodiment, the present invention provides a recursive latent diffusion model (RLDM) for molecular dynamics. For instance, regarding FIGS. 3-7, there might not be any visual differences between this RLDM and the above. But, in some embodiments, RLDM can be associated with modeling of the conditional distribution as an additive model and the gradient of a potential function. In molecular dynamics, the sequence is generated where ƒ_{θ} or g_{θ} represent the integrator with a surrogate model for energy and forces as follows:

$$\\ {x}_{t + 1} = {x}_{t} + ϵ {\partial }_{x} U ( {x}_{t} )$$

where U(x) is the potential and ∈ the integration step.

For the forward step, the following sequence can be used:

$$\\ \begin{matrix}{x}_{t + 1}^{{}_{} 0} \vert {x}_{t}^{{}_{} 0} + ϵ {\partial }_{x} {U}_{θ} ( {x}_{t}^{{}_{} 0} ) \\ {{⋮}_{}}^{} \\ {x}_{t + 1}^{{}_{} K} \vert {x}_{t}^{{}_{} K} + ϵ {\partial }_{x} {U}_{θ} ( {x}_{t}^{{}_{} K} )\end{matrix}$$

where xₜ₊₁^{k}|xₜ^{k}+∈∂ₓU_{θ}(xₜ^{k}) is the state xₜ₊₁^{k} conditional to the previous state xₜ^{k} plus the gradient of the potential ∂ₓU_{θ} at the previous state.

This model can be expanded in both time and diffusion directions, but requires to store a large number of variables. For training, it is possible to sample k and then unroll in time the sequence.

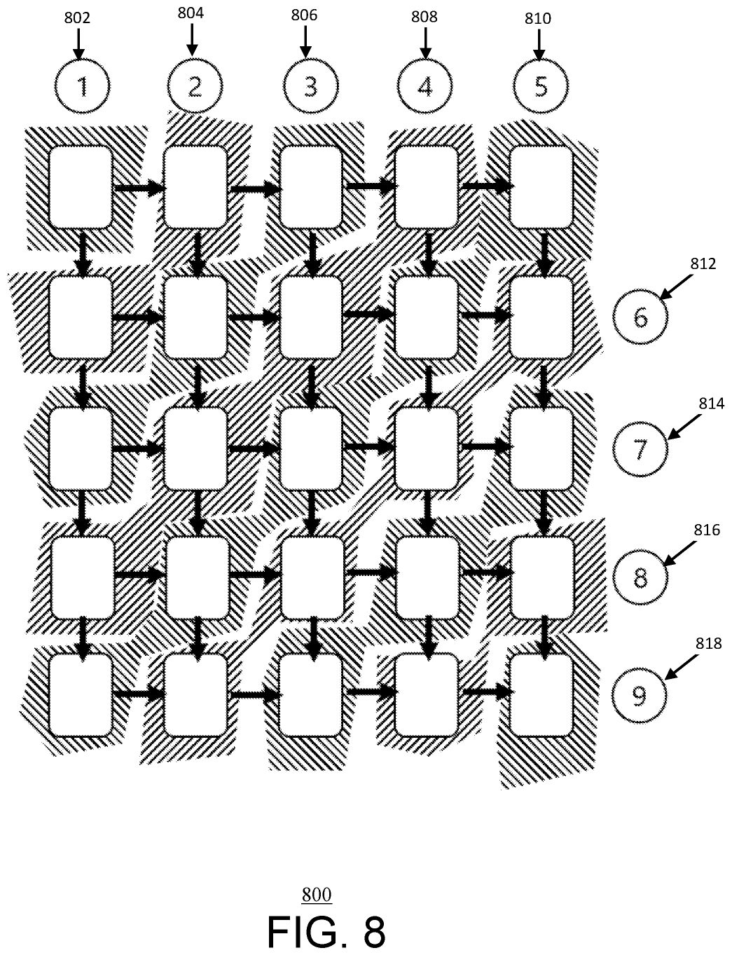

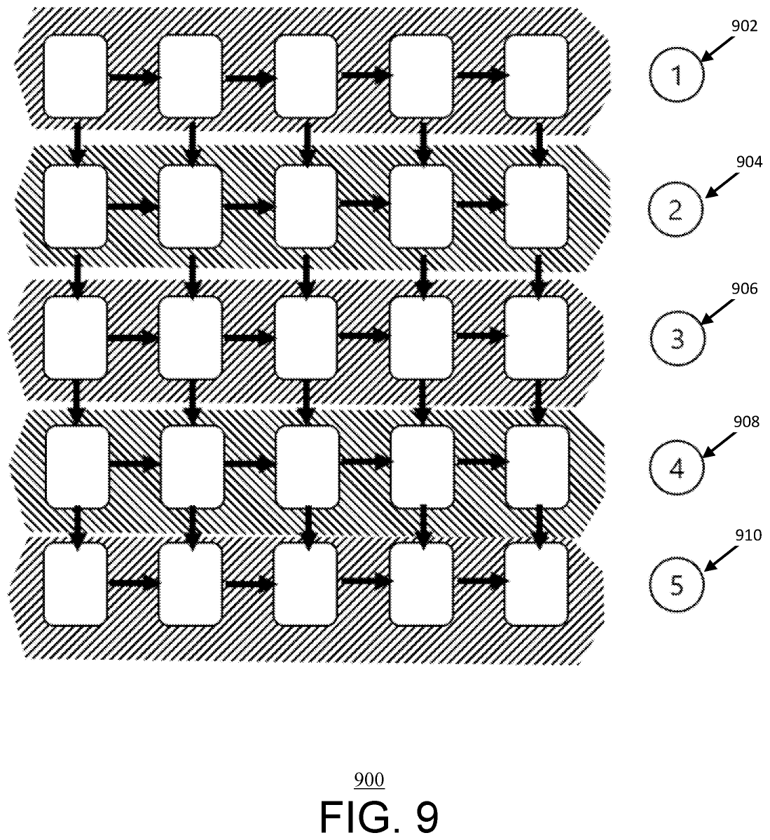

With respect to generation order complexity, in order to reduce the memory requirement, the method proceeds by generating all t=0, then generating all t=1 and keeping from t=0 only (t=0, k=K) the last point (see FIG. 8). The total memory is then K+T, where T is the length of the sequence. Alternatively, it is possible to run the forward (or backward) process in time t=0, . . . , T and then run in the diffusion direction k=0, . . . , K (see FIG. 9). In this case the memory requirement is T.

FIG. 8 illustrates the training sequence 800 for a sequential diffusion model according to an embodiment of the present invention. For instance, as mentioned before (e.g., FIGS. 3-6), a computing device may perform a method by generating each of the states (e.g., from the initial condition or state to each state along the diffusion/denoising direction as well as the temporal direction). Generating each of the states separately (e.g., each block from FIGS. 3-6) may use a significant amount of memory. To reduce the memory requirement, the computing device can perform the process describe in the training sequence 800. For instance, the computing device determines or generates the states in a group (e.g., groups 802-808). For instance, the computing device first generates the initial state (e.g., x₀⁰) for group 802. Then, the computing device generates two additional states (e.g., x₀¹ and x₁⁰) for group 804. The computing device continues through training sequence 800 to generate the rest of the states. By using the training sequence 800, the total memory is K+T, where T is the length of the sequence.

FIG. 9 illustrates the training sequence 900 for a sequential diffusion model according to another embodiment of the present invention. The training sequence 900 also trains or generates states in batches (e.g., batches 902-910). For instance, the computing device runs (e.g., generates) the process in forward or backward processes in time t=0, . . . , T, and then moves to each of the diffusion/denoising directions. For instance, the computing device starts with states for t=0 (e.g., group 902), then proceeds with t=1 (e.g., group 904), and so on. By doing this, the memory requirements become T.

An embodiment of the present invention provides for separate training of the forward and reverse models (ƒ_{θ}, g_{θ}, U_{θ}), and then using the diffusion model to model the initial condition and the boundary. These on the other side can be pre-trained on the input dataset conditions and then used as conditional generation of initial conditions, thus optimizing for x₀^{K} of the generation noise. For instance, the conditional models (e.g., ƒ_{θ}, g_{θ}, U_{θ}) can be trained separately using supervised loss (e.g., traditional supervised loss). Thus, first, a predictive model (e.g., a model that is not diffusion) can be trained. The predictive model can be, for example, a Neural Operator as FNO or a U-Net. Then, embodiments of the present invention can use this model as the conditional model for the diffusion model.

In some embodiments, an inverse problem with linear transformation is considered. For instance, an embodiment of the present invention considers the simplified problem of measuring x_{T} with linear dynamic:

xₜ=Hₜxₜ₋₁+nₜ

where n˜N(0, σ) is some noise in the dynamics (e.g., a normal distribution of noise with the mean being 0 and variance σ) and the measure is:

$$\\ y = {Hx}_{T} + n$$

with known noise level n˜N(0, σ) and measure matrix H, for an unknown input distribution p(x₀), where it is desired to sample from p(x₀|y), thus providing:

$$\\ {x}_{T} = {\prod }_{t = 0}^{T} {H}_{t} {x}_{0} + {\sum }_{t = 0}^{T} {\tilde{H}}_{t} {n}_{t}$$

with:

$$\\ {\tilde{H}}_{t} = {\prod }_{τ = t}^{T} {H}_{τ}$$

Where observing the system in the final step, the observation variable y can be written as a function of the final state as follows:

$$\\ y = \overline{H} {x}_{0} + \overline{n}$$

which is still a linear system of the initial state and where:

$$\\ \overline{H} = H {\prod }_{t = 0}^{T} {H}_{t}$$

and the noise is distributed according to:

$$\\ \overline{n} \sim N ( 0 , \overline{σ} {\overline{σ}}^{{}_{} T} ) , \overline{σ} = H {\sum }_{t = 0}^{T} {\tilde{H}}_{t} {σ}_{t}$$

If the dynamics of the system are not linear, then an embodiment of the present invention uses the previous relationship in an interval around the current state and uses the Taylor expansion of the operator as ƒ(x)≈ƒ(x₀)+∇ₓƒ(x)(x−x₀) or using the Euler integrator xt=xₜ₋₁+∈∇ₓU(xₜ₋₁) or xₜ=xₜ₋₁+∈Hxₜ₋₁, with H=∇ₓƒ(xₜ₋₁).

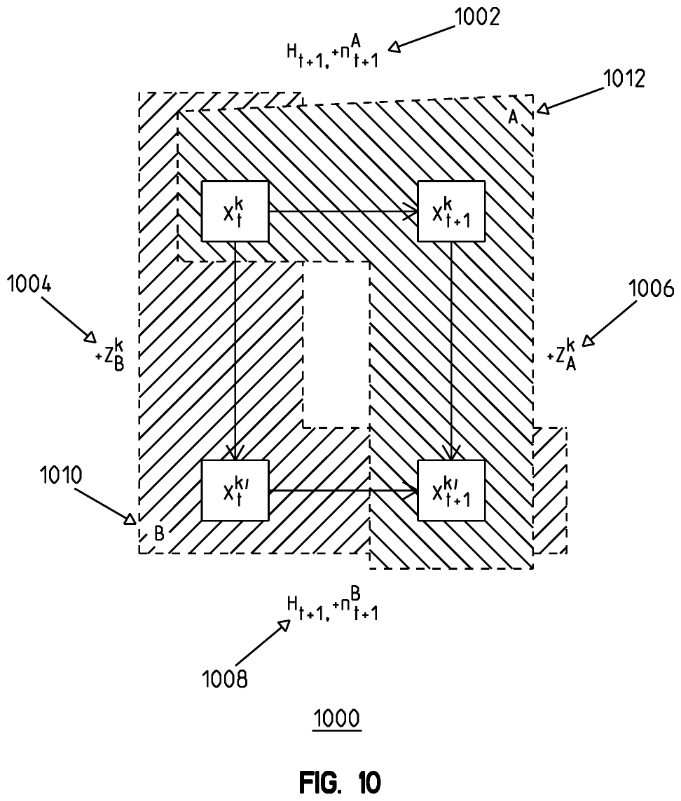

For SDM with linear transformation, the below is considered. For example, a linear transformation is considered along the time direction, with only the diffusion noise along the diffusion direction. Two paths are then considered in the diagram (see FIG. 10) with two consecutive time steps t, t+1 and two diffusion steps k′=k+1. For the diffusion steps, it is possible to write the close form of the noise for any k, k′. For the time direction, it is possible to write the noise variance using the results from the previous section as follows:

$$\\ {x}_{t} = {\hat{H}}_{t} {x}_{0} + {\tilde{n}}_{t}$$

with ñₜ=Σ_{τ=0}^{t}\tilde{H}_{τ}nₜ, \tilde{H}ₜ=Π_{τ=t}^{T}H_{τ}, Ĥₜ=Π_{τ=0}^{t}H_{τ}

Thus, the diagram shown in FIG. 10 can be used and it is possible to write the process xₜ₊₁^{k′} along the paths A and B. The path A can thus be written as follows:

$$\\ {x}_{t + 1}^{{}_{} {k}^{′} [ A ]} = {\hat{H}}_{t + 1} {x}_{t}^{{}_{} k} + {n}_{t + 1}^{{}_{} A} + {z}_{A}^{{}_{} k}$$

while the path B can be written as follows:

$$\\ {x}_{t + 1}^{{}_{} {k}^{′} [ B ]} = {\hat{H}}_{t + 1} {x}_{t}^{{}_{} k} + {n}_{t + 1}^{{}_{} B} + {z}_{B}^{{}_{} k}$$

where, while the transformation of the input is the same, the noise is different by walking along the two different paths because in the path B, one of the noise terms undergoes the linear transformation. This result shows also that the sequential diffusion model reduces to a diffusion model when the transformation is linear and equal to the identity transformation, which would be expected, while in the other cases, the sequential diffusion model requires the model to denoise a not-white noise, thus requiring the model to be aware to the dynamics of the system, as required. For instance, the above can refer to the linearized version. If the transformation is not linear, then embodiments of the present invention can linearize it. This model can be a special case where information is provided since this model can be used for the restoration of corrupted measures for sequences of data.

For instance, FIG. 10 illustrates a diagram for the inverse problem with linear transformation according to an embodiment of the present invention. For example, traversing along path A 1012, the above equation is determined as Ĥₜ₊₁xₜ^{k}+nₜ₊₁^{A} 1002 is added to z_{A}^{k} 1006. Traversing along path B 1010, the above equation is determined as Ĥₜ₊₁xₜ^{k}+nₜ₊₁^{B} 1008 is added to z_{B}^{k} 1004.

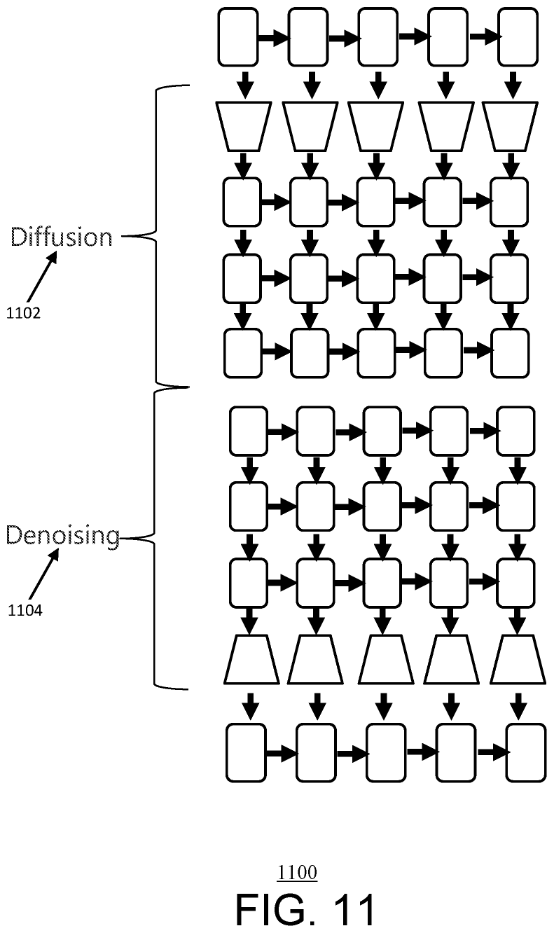

FIG. 11 illustrates a method for performing diffusion and denoising in a latent space according to an embodiment of the present invention. Referring to FIG. 11, a latent version can be implemented according to an embodiment of the present invention. The latent version uses an initial (encoder) network and final (decoder) network, and then performs the diffusion, prediction and denoising in the latent space.

For example, FIG. 11 shows the initial (encoder) network (e.g., the diffusion) 1102 and the final (decoder) network (e.g., denoising) 1104. The diffusion, prediction, and denoising can occur in the latent space. For instance, this model 1100 represents the use of latent variables. Latent variables are used to reduce the complexity of the computations. FIG. 11 shows the encoder 1102 and decoded mapping that takes the original input and maps it to the latent space, and back into the original space. For example, similar to FIGS. 3-6, FIG. 11 shows the diffusion (training) and denoising (generation) processes. In some embodiments, such as the protein case, embodiments of the present invention can model a large protein using a coarse-grained model of the system, e.g., less number of atoms by grouping using a pooling mapping to a smaller system.

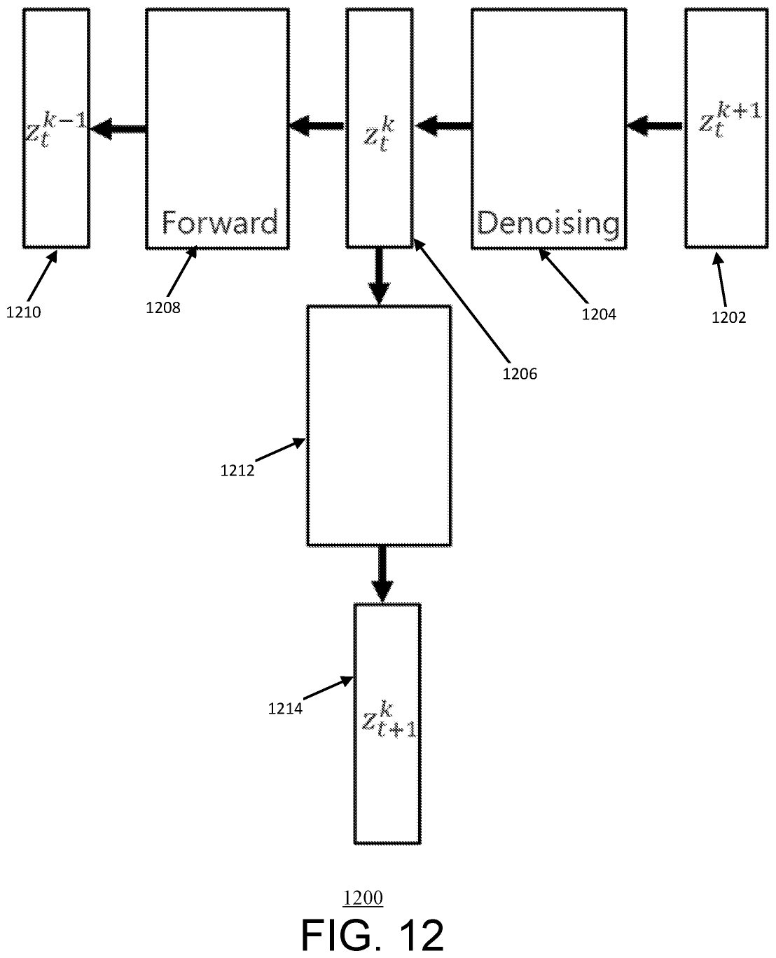

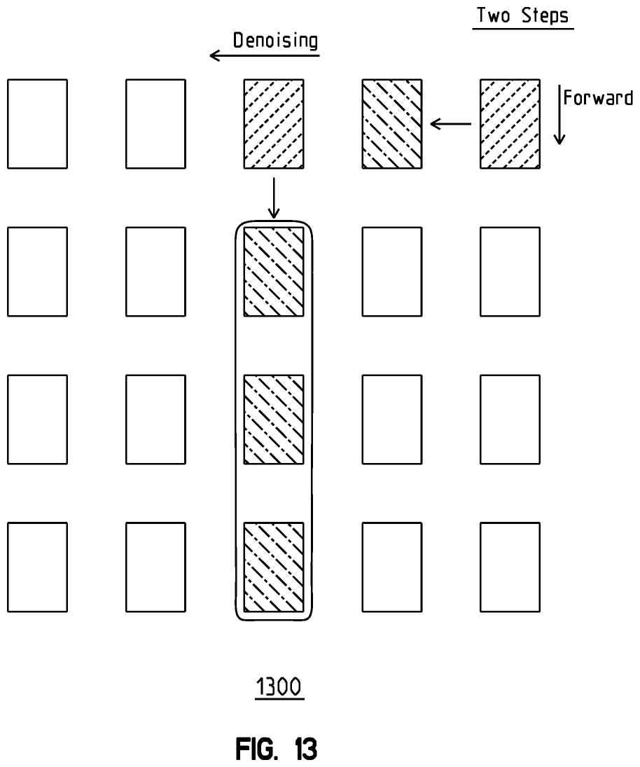

Embodiments of the present invention provide for denoising in one step or in two steps. In particular, when implementing the method according to embodiments of the present invention, it is possible to either separate the denoising and the forward operator learning or have the same architecture to do both steps. In the two-step implementation (see FIGS. 12 and 13), there are two networks that perform the two steps: 1) denoising and 2) forward prediction, where the forward prediction can also consider multiple steps ahead.

For example, FIG. 12 illustrates a method with a two-step implementation of denoising and forward prediction according to an embodiment of the present invention and FIG. 13 is a visualization of the two-step implementation according to an embodiment of the present invention. For instance, referring to FIG. 12, the method 1200 includes states 1202-1214 (e.g., values/variables). For instance, FIG. 12 shows more details regarding the network and the variables (e.g., input/output). State 1202 represents the state (zₜ^{k+1}) at real time k+1 at diffusion time t, then it is denoised by state 1204 to move to state 1206 (zₜ^{k}), which is the denoised version. Then, state 1208 shows the forward process 1208, which generates the result at state 1210 (zₜ^{k-1}). State 1212 shows another transformation to state 1214 (zₜ₊₁^{k}). Referring to FIG. 13, the visualization 1300 represents when to either denoise or to predict multiple time steps.

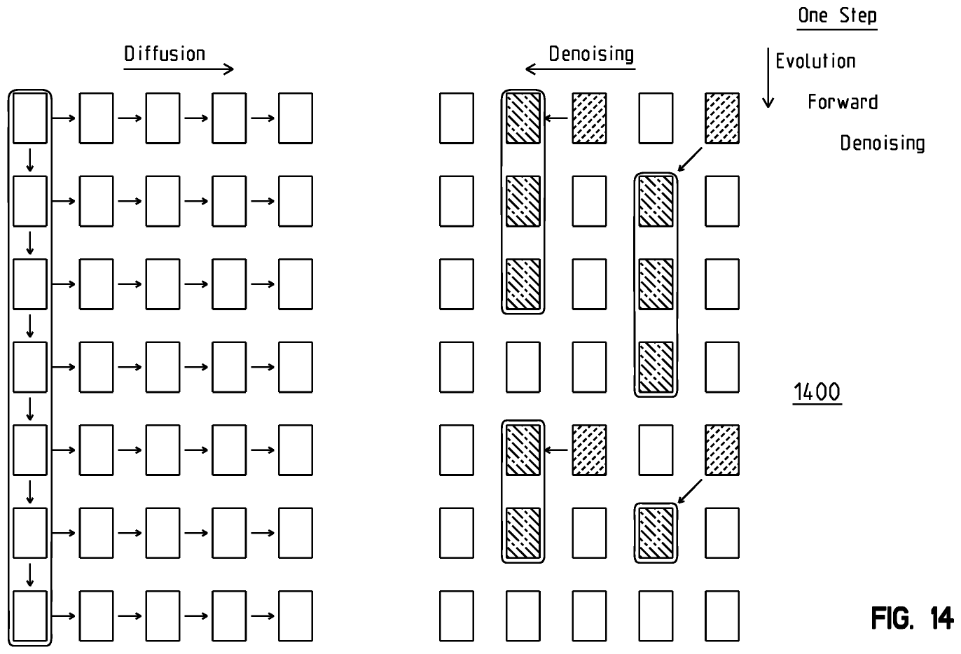

In the one-step implementation (see FIG. 14), there is only a single network that performs at the same time the two steps: 1) denoising and 2) forward prediction, where the forward prediction can also consider multiple steps ahead. For example, FIG. 14 illustrates a method 1400 with a one-step implementation and a visualization of the one-step implementation, and shows multiple implementations that consider: one step forward, one forward and one at the same time; one at the same and two forward, or three steps forward. For instance, method 1400 shows variation, where prediction and denoising can be performed at the same time. Or, first predicting all the sequences that can be performed, and then denying the full sequence.

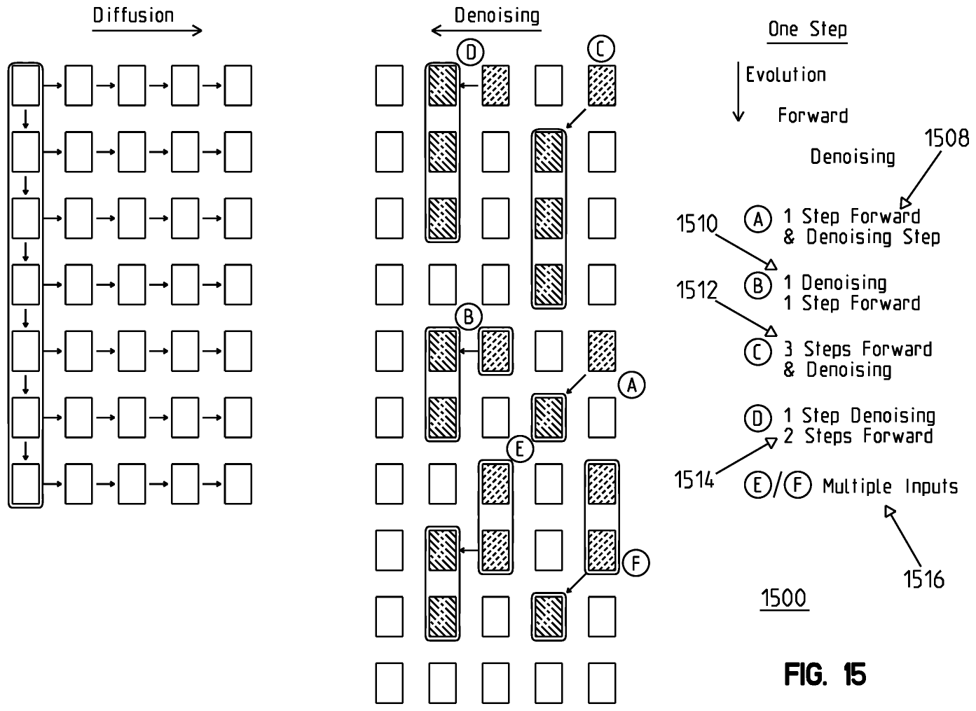

FIG. 15 illustrates multiple configurations of a forward denoising network according to an embodiment of the present invention. For instance, as shown in FIG. 15, various configurations 1500 are possible, where multiple inputs and outputs are considered. The inverse problem can be implemented by conditioning on the observed value. For instance, FIG. 15 shows some alternative examples. For example, at step 1508: in one step, prediction and denoise are performed. At step 1510, two time steps are denied (e.g., 1 denoising and 1 step forward). At step 1512, denoise is performed 3 steps in the future. At 1514, denoise one step in the present and two step in the future is performed. At step 1516, two steps to denoise/predict (one or two steps in time) are used.

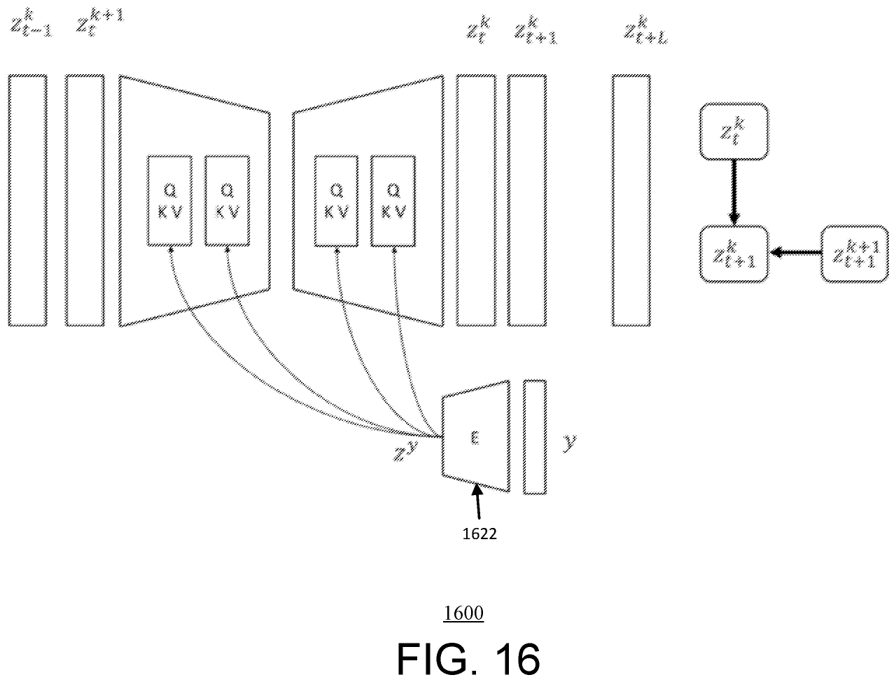

FIG. 16 illustrates a forward denoising neural network according to an embodiment of the present invention. For example, FIG. 16 shows a denoising auto-encoder architecture 1600 where the conditional information is encoded using a key-value-query procedure (e.g., self-attention). The conditional information is first encoded with a separate network E 1622. The denoising auto-encoder architecture can have multiple inputs and multiple outputs, which is described above.

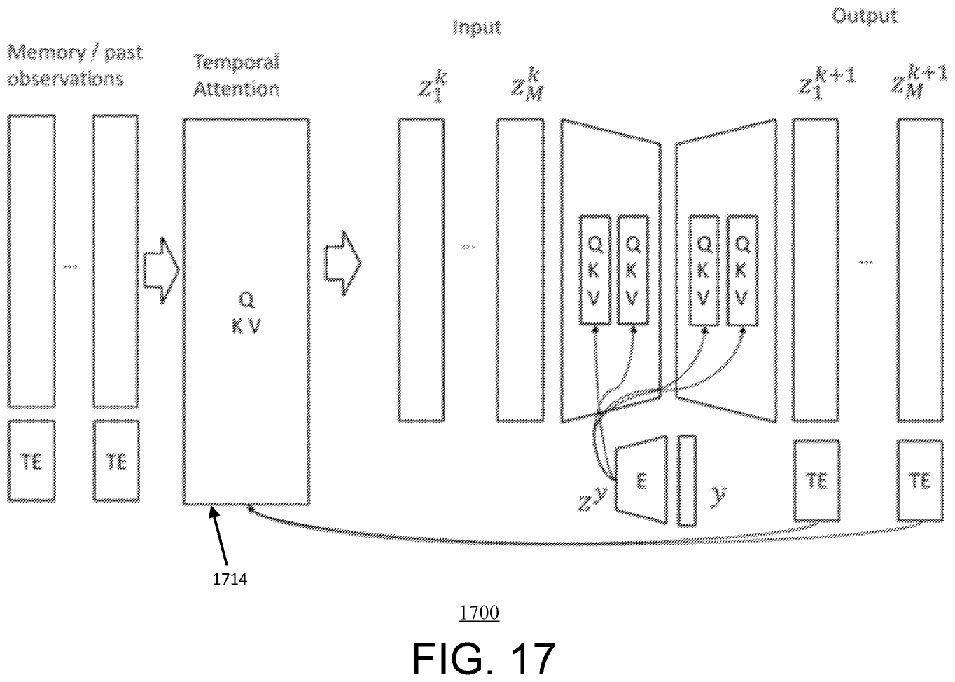

FIG. 17 illustrates a temporal attention and temporal encoding for infinite memory and look-ahead according to an embodiment of the present invention. Referring to FIG. 17, an embodiment of the present invention provides for infinite memory and look-ahead with temporal encoding and temporal attention. Here, a model is considered with potentially unlimited memory and/or look-ahead. First, a neural network is considered with K input and one output. A temporal encoding of the past observations is also provided. This temporal encoding is used to generate the M samples using an attention mechanism. Similarly, it is possible to use the output temporal encoding and an additional input for the temporal attention mechanism to generate the potentially infinite look-ahead predictions. An estimation of the error or noise level is also trained as additional output. For example, FIG. 17 shows the temporal attention. This can indicate that the prediction can happen in any time step in the future. This is achieved with an additional self-attention block 1714 working in the time dimension.

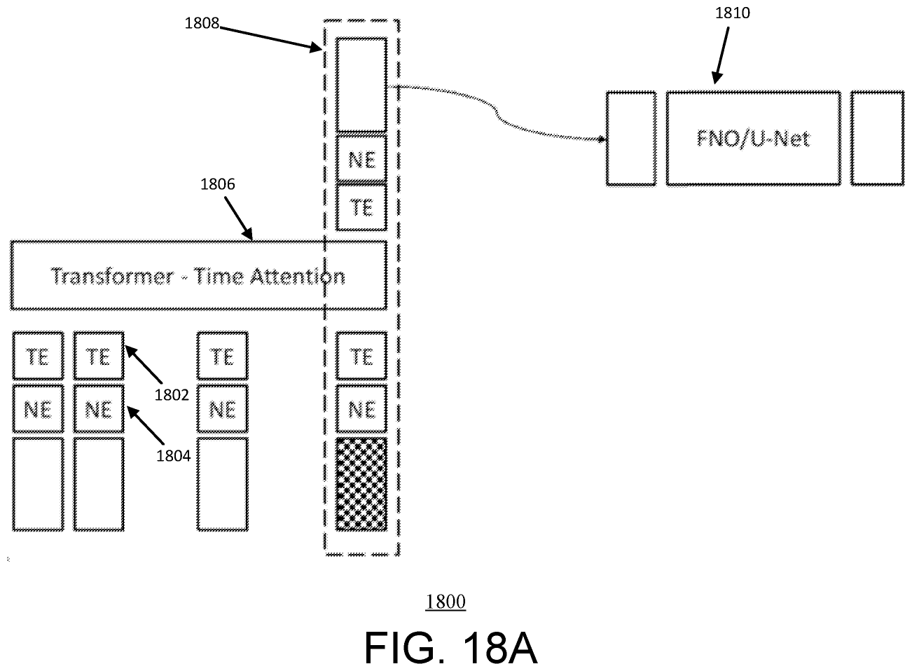

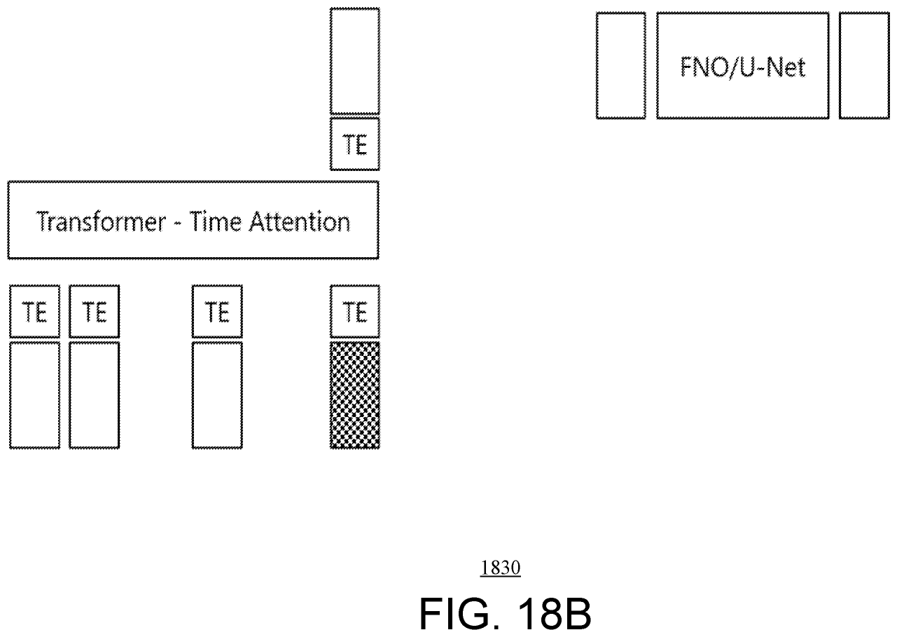

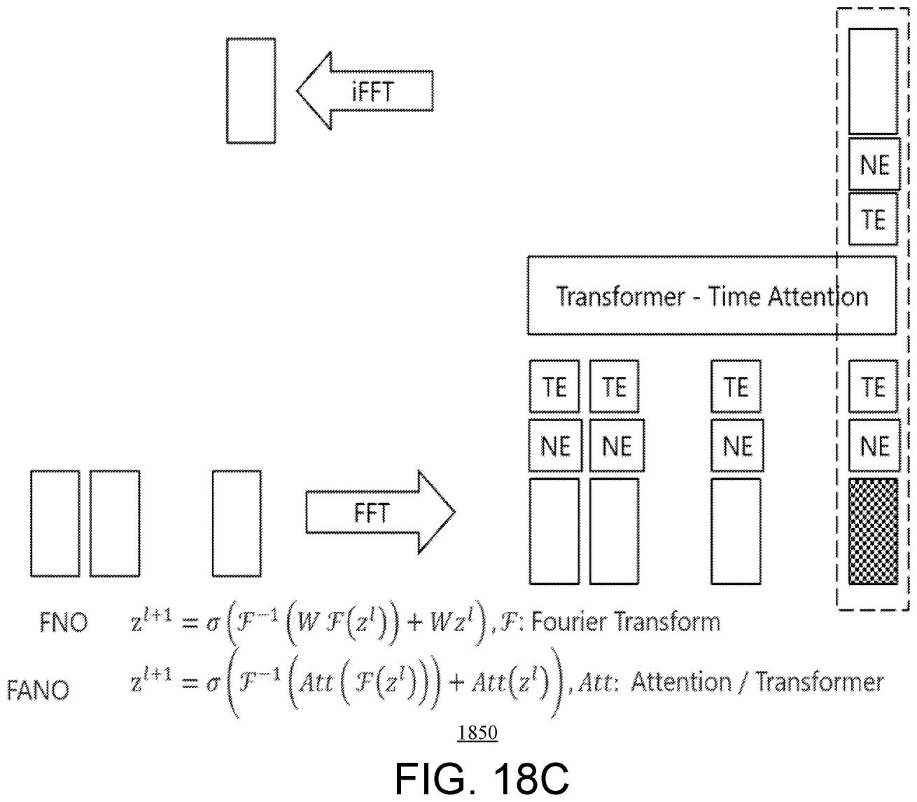

FIGS. 18A-C illustrates details of the implementations 1800, 1830, and 1850 of the time attention, and additional encoding that is used for the noise, the noise encoding (NE) 1804 and the time encoding (TE) 1802 according to an embodiment of the present invention. For instance, with respect to time and noise attention, FIG. 18A shows how the attention mechanism works, where the query point is the right element 1810 and the transformer-time attention block 1806 is shown on the left. The output is then used in the FNO/U-Net network. For example, FIGS. 18A-C represent the use of the temporal attention and the encoding of the forward model using FNO/U-Net. The dotted line 1808 of FIG. 18A highlights the same time step.

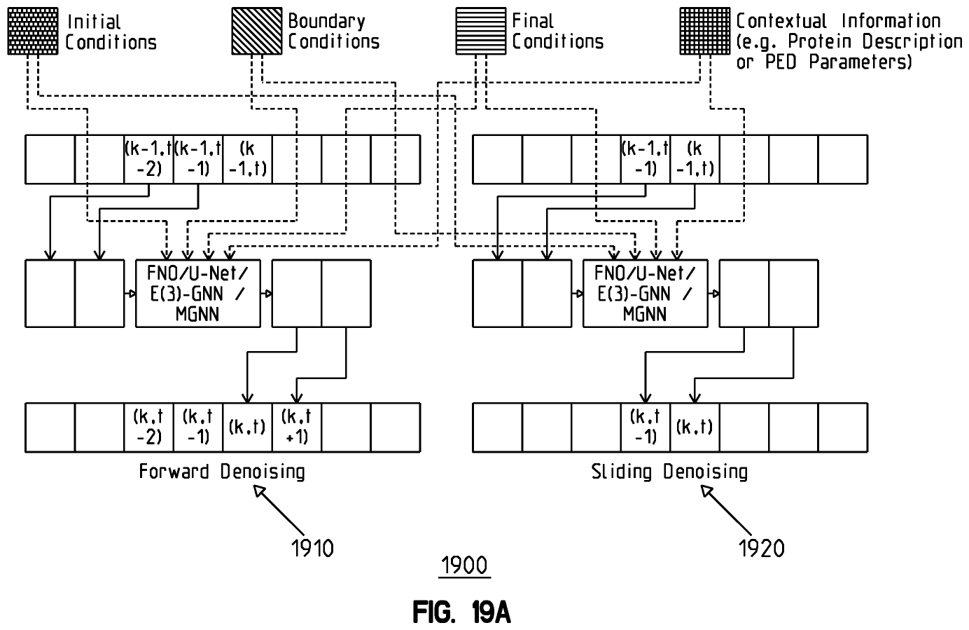

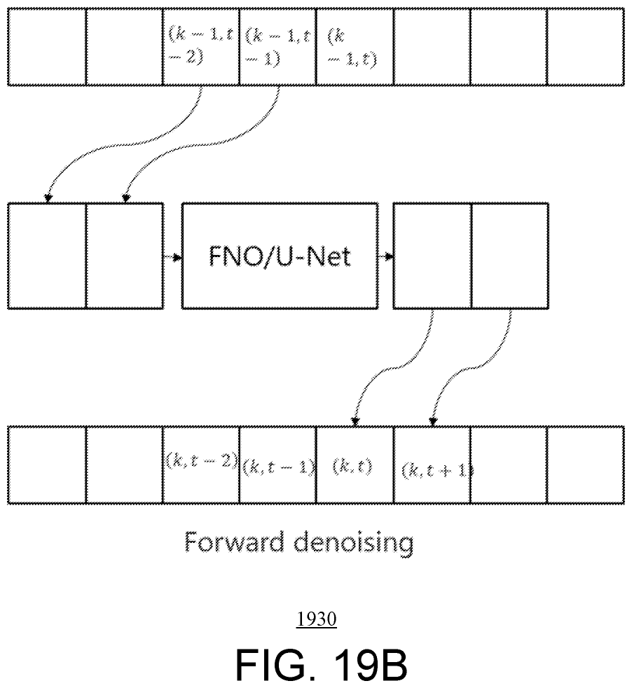

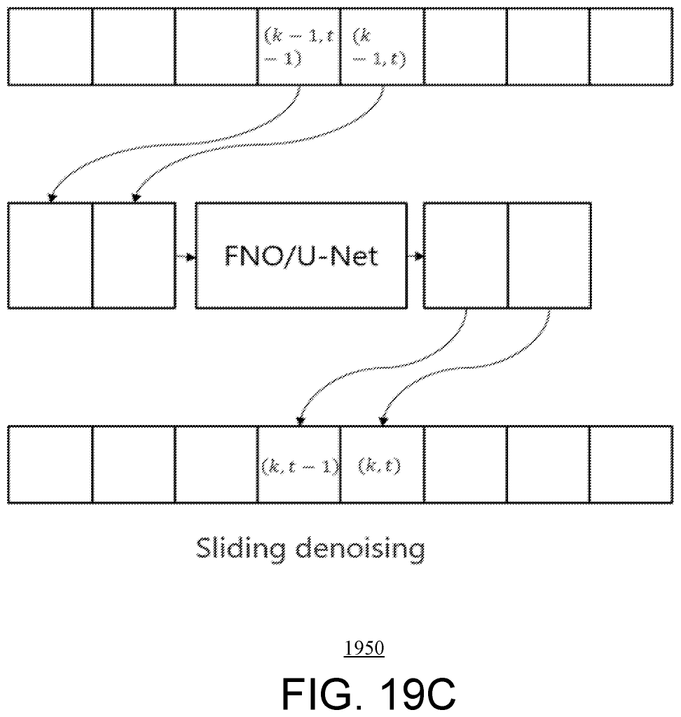

FIGS. 19A-19C illustrate an efficient denoising process (forward and sliding) according to an embodiment of the present invention. Referring to FIG. 19A, an embodiment of the present invention provides for a particularly memory efficient implementation. Here, two efficient denoising processes are considered: the forward denoising 1910 and the sliding denoising 1920. In the forward model, a neural network is used to predict the denoised version of the input, while in the sliding model, a window of the input is used and the same window is denoised in the diffusion direction k−1 to k. The input and output windows can have different sizes and the advance could depend on the output length. The encoder and decoder of the latent process could be trained separately as a standard PDE/MD training procedure, while the time evolution is generated in the latent space by the diffusion model. For instance, FIGS. 19A-19C are variations 1900, 1930, and 1950 of the same approach, where embodiments of the present invention can have multiple input/output. FIG. 19A shows that the input can be the initial condition, the boundary condition, the final condition (inverse problem), or other contextual information for the forward denoising 1910 and the sliding denoising 1920.

Embodiments of the present invention provide for dynamic and/or static conditional diffusion models. In particular, when generating and training conditional diffusion models there are conditioning variables, which can be:

1) Static (as for example with external information as measures or initial condition or boundary conditions).
2) Dynamic (as for example the previous time steps).

The static variable(s) does not change during test time, while the dynamic variable(s) depends on the current generated time step.

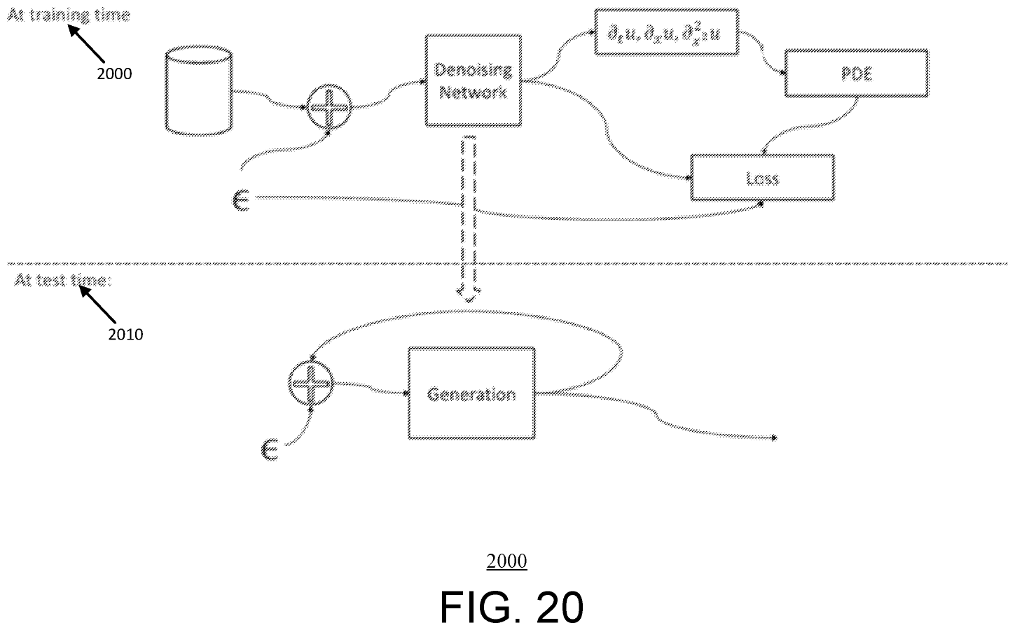

With respect to physics loss training, when the underlying partial differential equation is known, a loss is added that measures the difference between the predicted denoised version and the evaluation of the PDE. In this regard, reference is made to Li, Zongyi, et al., “Physics-informed neural operator for learning partial differential equations,” arXiv:2111.03794 (Nov. 14, 2022), which is hereby incorporated by reference herein. FIG. 20 illustrates a physical loss to train the network to follow the physical law during the training of the denoising network according to an embodiment of the present invention, where c is some error. For instance, FIG. 20 represents a method 2000 for how to use the implicit function (physics-informed neural network (PINN)) to implement SDM. The upper part 2000 represents the training, where the network is trained with respect to a loss and a mathematical description of the problem (or some data) in a supervised way. The generation step is shown in the lower part 2010.

Embodiments of the present invention can be practically applied to effect further improvements in various technical fields of application, such as automated medicine, drug, molecule or material discovery, catalyst design, genetic sequencing, seismic wave inversion, aerodynamic design, video restoration, and other machine learning tasks using simulations of physical systems.

One practical application is for drug discovery, protein-ligand binding predictions, and/or molecule generation from fragments. For molecule generation, the sequential diffusion model according to an embodiment of the invention models binding of molecules fragments where the evolution is modelled as a molecular dynamic. In this context, the input are the proteins or molecular segments to represent, for example either in the simplified molecular-input line-entry (SMILE) format or directly as a 3D point cloud, or alternatively, directly as output of accurate molecular dynamics models such as AlphaFold. The output will be the final configuration (position of atoms) and possibly additional elements (linkers) described as atoms and coordinates. The output can include physical properties such as energy, forces, potential energy function, if molecules bind, etc. For the drug discovery or molecular dynamic applications, an E(3)-GNN (graph neural network) or MGNN (multimodal graph neural network) is used that is rotation and translation equivariant for the denoising process.

Another practical application is for catalyst design. Here, the sequential diffusion model according to an embodiment of the invention models the process of catalysis with an absorbent and is used to predict forces, as well as energy and properties of the chemical reaction. The inputs are sequences of configurations (trajectories) of the molecules (atom type, atom coordinates, energy and forces) and the output are trajectories for new initial configurations. From the trajectories, free energy of the final configurations and energy distribution are derived.

Another practical application is for genetic sequence modelling. Here, the sequential diffusion model according to an embodiment of the invention models the genetic sequence of amino acids, where the forward model is a standard recursive neural network, such as a long short-term memory (LSTM) or gated recursive unit (GRU). In this case, there is a sequence of genetic encoded sequences, including genetically modified versions, with information on external factors. New sequences are then generated, conditioned to new external factors.

Another practical application is for seismic wave inversion, such as for oil and gas discovery or underground/sea analysis. Here, the sequential diffusion model according to an embodiment of the invention models the inverse wave equation problem when a wave equation propagates in a medium, for example for gas and oil exploration or for analysis of ground composition. As training data, wave measurements are used, also from simulated data conditioned to material and boundary conditions. At test time, the configuration (material and boundary conditions) that justifies the observed measures is found. Alternatively, it is possible to train to directly predict the material composition conditioned to the observations and then generate multiple possible configurations.

Another practical application is for aerodynamic design. Here, the sequential diffusion model according to an embodiment of the invention models the flow of air (gas) on surfaces of vehicles to design the shape of the vehicle itself, as for example the airfoil of the wings of an airplane. In this case, training data is provided from simulation or real experiments conditioned to the shape and boundary conditions of the airplane/airfoil. Once the model is trained, it is used to generate new forward simulation or to form (desired) observations to generate the boundary conditions. This can be used to predict the air flow, reconstruct the airplane configuration (e.g., from the black-box in case of emergency) or to help the wing or airfoil design and optimization.

Another practical application is for video restoration, in particular for denoising video sequences. In this case, the previous image can be modeled as optical flow. The model is trained on various video sequence, then at test time, the video sequence is denoised by running the diffusion generation process conditioned to the observation (either a noisy version of the video, or the initial or last frame, or intermediate frames).

In an embodiment, the present invention provides a method comprising the following steps:

1) Collect the data for training.

a. Additionally or alternatively, simulated data is used, for example by iteratively requesting a numerical simulator to generate new data (based on the performance of the model) in an active learning cycle.

b. For molecular data, the input data can be described in the SMILE format. The data contains molecules (as proteins) with their configuration (location in 3D space) and atom types. The position can also be represented in 2d coordinates. In addition, either the output of a highly accurate molecular dynamics model or the ability to run an accurate molecular dynamics model on demand (for the active training scenario) is used to provide the training data for the model.

2) Train the denoising model conditioned to the initial conditions, boundary conditions, observations or final conditions, but also conditioned to the past or future steps (context).

a. The training occurs by sequentially and recursively updating the model in order to predict the noise or the clean input, after adding Gaussian noise to the input (denoising model training) at each step in the diffusion direction and training a denoising network to reconstruct the noise. This starts from a noisy version of the input and then denoising is done conditional to the input. Part of the input can be fixed and never modified, while new components can be generated. At every diffusion step, the denoised sequence is generated by propagating the sequential process (unroll) either in the forward or backward direction (depending on the problem), where each propagation step gets the conditioning from the previous time step and conditioning on the input (e.g., initial condition or observation) and some variable to optimize upon (e.g., boundary conditions or the initial condition when training in forward, but desiring to use the model to solve the inverse problem). For instance, this is described above. For example, FIG. 19A shows various conditional inputs while FIGS. 3-6 show the condition on the previous time step. The process follows the following steps:

b. Use of a sequential diffusion model to model the forward and inverse problem for the simulation of a physical system with applications, e.g., to drug discovery, seismic and aerodynamic systems.
i. A sequential model where the condition is on a forward or reverse model which are modelled with neural networks.
ii. Where the boundary condition is model as a discrete variable.
iii. Where for modelling molecular systems, the condition is an integrator based on the gradient of a potential energy which is modeled using a neural network.
iv. Where physical constraints and physical laws are used as loss to minimize the denoised prediction, such that it is physical consistent or follows some given condition (e.g., when generating video by using a language model and the description of the video in words or sentences, or the textual description for consecutive frame changes according to an external given distance).
   3) Generate, e.g., the sequence of molecule configurations, solution of PDE or video.

a. Use the conditional or sequential diffusion model to generate either the forward or the inverse solution, where for the inverse problem the input is the measure of the final time, and for the forward problem the input is the initial condition.

b. For solving for the boundary condition, multiple forward sequences are simulated and the gradient is accumulated to find the optimal boundary conditions. In this phase, the user can input high level descriptions of the molecules (SMILE) which are then converted in the 3D format, or directly the 3D configuration. The user then gets as an output the 3D description which can be converted in the SMILE format. The user can also input the desired properties of the generated molecule, which will be used during the generation process to produce the expected property.

c. The model conditioned to the past or future solutions and to external input, as for example the parameters of the PDE or the material to simulate, the observations, initial conditions, final conditions, boundary conditions.

Embodiments of the present invention provide for the following improvements over existing systems:

1) Use of a sequential diffusion model, where the diffusion direction and the time direction are modelled separately, to model the forward and inverse problems for the simulation of a physical system with applications, e.g., to drug discovery, seismic and aerodynamic systems.

a. A sequential model where the condition is on a forward or reverse model which are modelled with neural networks.

b. Where the boundary condition is model as a discrete variable.

c. Where for modelling a molecular system, the condition is an integrator based on the gradient of a potential energy which is modeled using a neural network.

d. Where the training is done by minimizing the error with respect to some given physical laws (in term of potential or PDE) or some external information (such as the text description).

2) The method can model the conditional distribution p(x|y) where x is the set of sequences and y is an external information. The model extends state of the art systems for machine learning models to dynamic systems, thus bringing the same or improved accuracy and generalization capabilities.
3) With respect to alternative diffusion models and systems for learning and using the models, the approach according to embodiments of the present invention reduces the memory requirements, making the training possible in current systems.

a. Reduces required training memory relative to current approaches and is computationally more efficient, and thereby saves computational power and resources.

b. Enables to model complex data distributions.

In an embodiment, it is possible to train sequences with zero correlation between samples and still be able to predict correctly the output. In an embodiment, the input could be the description of fragments and optionally the property required. The output in this case is the final configuration, the additional required fragments/linker and properties as binding/not binding. In an embodiment, it is enabled to model a stochastic system. In an embodiment, the system is able to generate multiple solutions.

The following references are hereby incorporated by reference herein:
Rombach, Robin et al., “High-Resolution Image Synthesis with Latent Diffusion Models,” Computer Vision and Pattern Recognition, arXiv:2112:10752 (Apr. 13, 2022).
Igashov, Ilia, et al., “Equivariant 3D-Conditional Diffusion Models for Molecular Linker Design,” Machine Learning, arXiv:2210.05274 (Oct. 11, 2022).

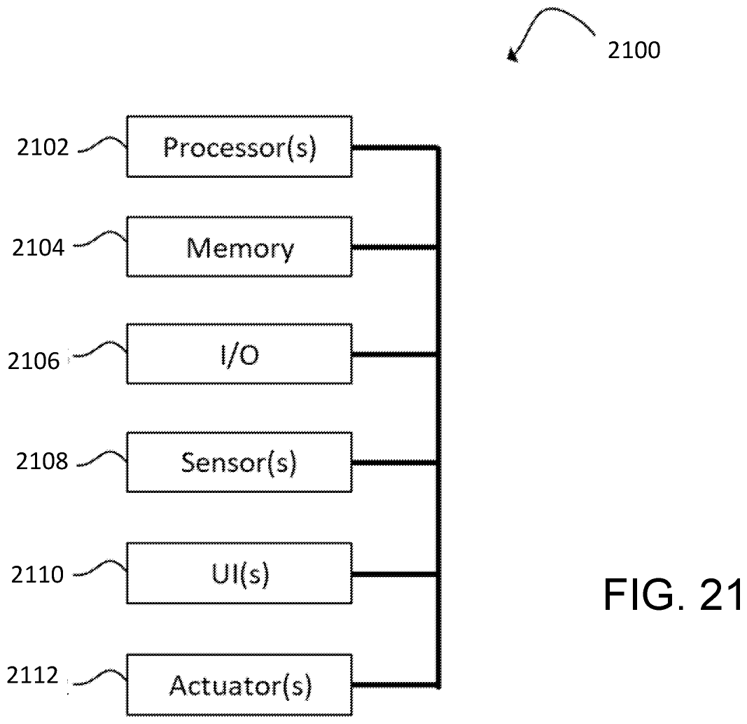

FIG. 21 is a block diagram of an exemplary processing system, which can be configured to perform any and all operations disclosed herein. For instance, a processing system 2100 can include one or more processors 2102, memory 2104, one or more input/output devices 2106, one or more sensors 2108, one or more user interfaces 2110, and one or more actuators 2112. Processing system 2100 can be representative of each computing system disclosed herein.

Processors 2102 can include one or more distinct processors, each having one or more cores. Each of the distinct processors can have the same or different structure. Processors 2102 can include one or more central processing units (CPUs), one or more graphics processing units (GPUs), circuitry (e.g., application specific integrated circuits (ASICs)), digital signal processors (DSPs), and the like. Processors 2102 can be mounted to a common substrate or to multiple different substrates.

Processors 2102 are configured to perform a certain function, method, or operation (e.g., are configured to provide for performance of a function, method, or operation) at least when one of the one or more of the distinct processors is capable of performing operations embodying the function, method, or operation. Processors 2102 can perform operations embodying the function, method, or operation by, for example, executing code (e.g., interpreting scripts) stored on memory 2104 and/or trafficking data through one or more ASICs. Processors 2102, and thus processing system 2100, can be configured to perform, automatically, any and all functions, methods, and operations disclosed herein. Therefore, processing system 2100 can be configured to implement any of (e.g., all of) the protocols, devices, mechanisms, systems, and methods described herein.

For example, when the present disclosure states that a method or device performs task “X” (or that task “X” is performed), such a statement should be understood to disclose that processing system 2100 can be configured to perform task “X”. Processing system 2100 is configured to perform a function, method, or operation at least when processors 2102 are configured to do the same.

Memory 2104 can include volatile memory, non-volatile memory, and any other medium capable of storing data. Each of the volatile memory, non-volatile memory, and any other type of memory can include multiple different memory devices, located at multiple distinct locations and each having a different structure. Memory 2104 can include remotely hosted (e.g., cloud) storage.

Examples of memory 2104 include a non-transitory computer-readable media such as RAM, ROM, flash memory, EEPROM, any kind of optical storage disk such as a DVD, a Blu-Ray® disc, magnetic storage, holographic storage, a HDD, a SSD, any medium that can be used to store program code in the form of instructions or data structures, and the like. Any and all of the methods, functions, and operations described herein can be fully embodied in the form of tangible and/or non-transitory machine-readable code (e.g., interpretable scripts) saved in memory 2104.

Input-output devices 2106 can include any component for trafficking data such as ports, antennas (i.e., transceivers), printed conductive paths, and the like. Input-output devices 2106 can enable wired communication via USB®, DisplayPort®, HDMI®, Ethernet, and the like. Input-output devices 2106 can enable electronic, optical, magnetic, and holographic, communication with suitable memory 2104. Input-output devices 2106 can enable wireless communication via WiFi®, Bluetooth®, cellular (e.g., LTE®, CDMA®, GSM®, WiMax®, NFC®), GPS, and the like. Input-output devices 2106 can include wired and/or wireless communication pathways.

Sensors 2108 can capture physical measurements of environment and report the same to processors 2102. User interface 2110 can include displays, physical buttons, speakers, microphones, keyboards, and the like. Actuators 2112 can enable processors 2102 to control mechanical forces.

Processing system 2100 can be distributed. For example, some components of processing system 2100 can reside in a remote hosted network service (e.g., a cloud computing environment) while other components of processing system 2100 can reside in a local computing system. Processing system 2100 can have a modular design where certain modules include a plurality of the features/functions shown in FIG. 21. For example, I/O modules can include volatile memory and one or more processors. As another example, individual processor modules can include read-only-memory and/or local caches.

In the following, embodiments of the present invention are described in further detail, including the embodiments described above. Embodiments of the present invention aim to advantageously exploit a denoising diffusion model for solving forward, inverse, and/or molecular simulations (e.g., physical simulations). To achieve this objective, embodiments of the present invention model the underlying process as a sequence in forward (or reverse) time.

As part of the problem formulation, a sequence of variables x₀, . . . , x_{T} is considered. The sequential diffusion model is used to generate this sequence by estimating the probability density (p(x₀, . . . , x_{T})) from the data. While it may be possible to create an explicit model for the forward or reverse generation procedure, this has some drawbacks similar to drawbacks of the existing technology, in particular:
1. Vanishing gradients and an increase in error with the length of T.
2. Only generating the initial state, and all the sequence would be determined since the evolution is deterministic.

Embodiments of the present invention provide an alternative approach that does not suffer from these and other technical drawbacks. For instance, embodiments of the present invention provide a sequential diffusion model (SDM) or an alternatively recurrent denoising diffusion model. Embodiments of the present invention considers and/or uses a special form of conditional denoising diffusion model, where sampling occurs from a conditional diffusion model over the previous (and/or future) time step. This model can be practically applied to learn: 1) a neural operator for solving PDEs; 2) the inverse problem for PDEs; 3) the forward molecular dynamics problem (from a configuration to generate a stable configuration); 4) a diffusion model to generate a stable configuration from fragments of molecules; 5) a diffusion model to model sequences, as DNA/RNA/mRNA sequences; and/or 6) a diffusion model for video restoration.

Embodiments of the present invention can include learning forward and inverse recurrent denoising diffusion model for modeling PDE, molecular dynamic, and/or DNA/RNA sequences.

In contrast, an alternative approach that naively uses a diffusion model to generate the full sequence would be more computationally complex and would have increased computational cost because it would require a neural network that has the complete sequence as input and output, thus also limiting its use to a fixed length.

Diffusion models learn a data distribution p(x) from samples by iteratively denoising a normal distributed random variable, modeled as the reverse (generative) process of a Markov Chain of length K+1. The training is performed by minimizing a variational lower bound on the forward process p(x^{K})Π_{k=K-1}⁰p(x^{k-1}|p^{k}). These models are implemented as a sequence of denoising autoencoder ∈_{θ}(x^{k}, t); k=0, . . . , K that are trained to a denoised version of the perturbed input variable x^{k}, with a loss function:

$$\\ {ℒ}^{{}_{} DM} = {𝔼}_{x , ϵ \sim 𝒩 ( 0 , 1 ) , k} [ {‖ ϵ - {ϵ}_{θ} ( {x}^{{}_{} k} , k ) ‖}_{2}^{2} ]$$

where k∈[K] is uniformly sampled.

A latent diffusion model (LDM) is considered with latent variables z^{k}. Latent variables can be variables that cannot be observed. The loss function is:

$$\\ {ℒ}^{{}_{} LDM} = {𝔼}_{{ε}_{ϕ} ( x ) , ϵ \sim 𝒩 ( 0 , 1 ) , k} [ {‖ ϵ - {ϵ}_{θ} ( {z}^{{}_{} k} , k ) ‖}_{2}^{2} ]$$

with ε_{ϕ}(x) the trainable encoding function and [symbol] _{ϕ}(z) the decoder network.

A conditional latent diffusion model (CLDM) can be provided as follows:

$$\\ {ℒ}^{{}_{} CLDM} = {𝔼}_{{ε}_{ϕ} ( x ) , ϵ \sim 𝒩 ( 0 , 1 ) , k} [ {‖ ϵ - {ϵ}_{θ} ( {z}^{{}_{} k} , k , {τ}_{ψ} ( y ) ) ‖}_{2}^{2} ]$$

where τ_{ψ}(y) is a neural network with parameters ψ, that represents the encoding of the conditioning variable y.

A conditional latent neural network considers a U-Net, a convolutional network architecture, and a Fourier neural operator (FNO) with a cross-attention mechanism. The cross-attention layer of the

$$\\ U ‐ Net attention ( Q , K , V ) = softmax ( \frac{1}{√ ( d )} {QK}^{{}_{} T} ) V ,$$

with

$$Q = {W}_{Q}^{( l )} {z}^{( l ) k} , K = {W}_{K}^{( l )} {τ}_{ψ} ( y ) , V = {W}_{V}^{( l )} {τ}_{ψ} ( y )$$

and similar for the FNO, with the attention used to model the latent variable z^{(l+1)k}=attention(Q, K, V)(z^{(l)k}). The complexity can be lowered.

a partial differential equation (PDE) can then be considered in the form of:

$${δ}_{t} u ( x , t ) = {δ}_{x} u ( x , t ) + …$$

and the neural operator F:u(x, t−1)→u(x, t), thereby providing the sequence:

$$u ( x , t ) = F ( u ( x , t - 1 ) , B )$$

where u(x, 0) is the initial state and B represents the boundary conditions (e.g., constraints necessary for the solution of a boundary value problem). It is now possible to determine the CLDM for the exemplary PDE equation and its inverse.

Forward CLDM (F-CLDM): In order to generate the sequence at time t using the diffusion process, the state variable (xₜ^{k}) and conditional variable (yₜ^{k}) are set to:

$${x}_{t}^{k} = {u}^{k} ( x , t ) , {y}_{t}^{k} = {u}^{k} ( x , t - 1 )$$

An alternative conditioning function is a forward function ƒ_{θ}, which can be trained separately on the input sequence (thus giving a prior information to improve convergence) as follows:

$${x}_{t}^{k} = {u}^{k} ( x , t ) , {y}_{t}^{k} = {f}_{θ} ( {u}^{k} ( x , t - 1 ) )$$

Inverse CLDM (F-CLDM): Similarly, the inverse problem can be defined, where it is desired to generate the sequence for a time t using the diffusion process from t+1, as follows:

$${x}_{t}^{k} = {u}^{k} ( x , t ) , {y}_{t}^{k} = {u}^{k} ( x , t + 1 )$$

An alternative conditioning function is a backward function ƒ_{θ}, as follows:

$${x}_{t}^{k} = {u}^{k} ( x , t ) , {y}_{t}^{k} = {g}_{θ} ( {u}^{k} ( x , t + 1 ) )$$

where g_{θ}(u(x, t)) is the inverse propagation function.

As an alternative conditioning mechanism, the conditioning can happen also with a feature-wise linear modulation (FiLM) layer or the Taylor/channel conditioning.

An embodiment of the present invention models discrete variables as follows: 1) propagating the gradient using straight through; 2) using implicit maximum likelihood estimation (iMLE, i.e. perturb and MAP, see Pasquale Minervini, et al., “Adaptive Perturbation-Based Gradient Estimation for Discrete Latent Variables,” arXivL2209.04862 (Sep. 11, 2022), which is hereby incorporated by reference herein); 3) using one hot-encoding and then using the argmax; and/or 4) using a Markov chain in the discrete variable.

Thus, the forward sequential diffusion model and inverse sequential diffusion model become:

$${x}_{t}^{k} = {u}^{k} ( x , t ) , {y}_{t}^{k} = {f}_{θ} ( {u}^{k} ( x , t - 1 ) , B ) \\ {x}_{t}^{k} = {u}^{k} ( x , t ) , {y}_{t}^{k} = {g}_{θ} ( {u}^{k} ( x , t + 1 ) , B )$$

where the variables are concatenated.

In an embodiment, the present invention provides a recursive latent diffusion model (RLDM) for molecular dynamics. In molecular dynamics, the sequence is generated where ƒ_{θ} or g_{θ} represent the integrator with a surrogate model for energy and forces as follows:

$${x}_{t + 1} = {x}_{t} + ϵ {\partial }_{x} U ( {x}_{t} )$$

where U(x) is the potential and ∈ the integration step.

For the forward step, the following sequence can be used:

$${x}_{t + 1}^{0} \vert {x}_{t}^{0} + ϵ {\partial }_{x} {U}_{θ} ( {x}_{t}^{0} ) \\ ⋮ \\ {x}_{t + 1}^{K} \vert {x}_{t}^{K} + ϵ {\partial }_{x} {U}_{θ} ( {x}_{t}^{K} )$$

This model can be expanded in both time and diffusion directions, but requires to store a large number of variables. For training, it is possible to sample k and then unroll in time the sequence.

With respect to generation order complexity, in order to reduce the memory requirement, the method proceeds by generating all t=0, then generating all t=1 and keeping from t=0 only (t=0, k=K) the last point (see FIG. 8). The total memory is then K+T, where T is the length of the sequence. Alternatively, it is possible to run the forward (or backward) process in time t=0, . . . , T and then run in the diffusion direction k=0, . . . , K (see FIG. 9). In this case the memory requirement is T.

An embodiment of the present invention provides for separate training of the forward and reverse models (ƒ_{θ}, g_{θ}, U_{θ}), and then using the diffusion model to model the initial condition and the boundary. These on the other side can be pre-trained on the input dataset conditions and then used as conditional generation of initial conditions, thus optimizing for x₀^{K} of the generation noise.

In some embodiments, an inverse problem with linear transformation is considered. For instance, an embodiment of the present invention considers the simplified problem of measuring x_{T} with linear dynamic:

$${x}_{t} = {H}_{t} {x}_{t - 1} + {n}_{t}$$

where n˜N(0, σ) is some noise in the dynamics (e.g., a normal distribution of noise with the mean being 0 and variance σ) and the measure is:

$$y = {Hx}_{T} + n$$

with known noise level n˜N(0, σ) and measure matrix H, for an unknown input distribution p(x₀), where it is desired to sample from p(x₀|y), thus providing:

$${x}_{T} = {\prod }_{t = 0}^{T} {H}_{t} {x}_{0} + {\sum }_{t = 0}^{T} {\tilde{H}}_{t} {n}_{t}$$

with:

$${\tilde{H}}_{t} = {\prod }_{τ = t}^{T} {H}_{τ}$$

Where observing the system in the final step, the observation variable y can be written as a function of the final state as follows:

$$y = \overline{H} {x}_{0} + \overline{n}$$

which is still a linear system of the initial state and where:

$$\overline{H} = H {\prod }_{t = 0}^{T} {H}_{t}$$

and the noise is distributed according to:

$$\overline{n} \sim N ( 0 , \overline{σ} {\overline{σ}}^{T} ) , \overline{σ} = H {\sum }_{t = 0}^{T} {\tilde{H}}_{t} {σ}_{t}$$

If the dynamics of the system are not linear, then an embodiment of the present invention uses the previous relationship in an interval around the current state and uses the Taylor expansion of the operator as ƒ(x)≈ƒ(x₀)+∇ₓƒ(x)(x−x₀) or using the Euler integrator xₜ=xₜ₋₁+∈∇ₓU(xₜ₋₁) or xₜ=xₜ₋₁+∈Hxₜ₋₁, with H=∇ₓƒ(xₜ₋₁).

For SDM with linear transformation, the below is considered. For example, a linear transformation is considered along the time direction, with only the diffusion noise along the diffusion direction. Two paths are then considered in the diagram (see FIG. 10) with two consecutive time steps t, t+1 and two diffusion steps k′=k+1. For the diffusion steps, it is possible to write the close form of the noise for any k, k′. For the time direction, it is possible to write the noise variance using the results from the previous section as follows:

$${x}_{t} = {\hat{H}}_{t} {x}_{0} + {\tilde{n}}_{t}$$

with ñₜ=Σ_{τ=0}^{t}\tilde{H}_{τ}nₜ, \tilde{H}ₜ=Π_{τ=t}^{T}H_{τ}, Ĥₜ=Π_{τ=0}^{t}H_{τ}

Thus, the diagram shown in FIG. 10 can be used and it is possible to write the process xₜ₊₁^{k′} along the paths A and B. The path A can thus be written as follows:

$${x}_{t + 1}^{{k}^{′} [ A ]} = {\hat{H}}_{t + 1} {x}_{t}^{k} + {n}_{t + 1}^{A} + {z}_{A}^{k}$$

while the path B can be written as follows:

$${x}_{t + 1}^{{k}^{′} [ B ]} = {\hat{H}}_{t + 1} {x}_{t}^{k} + {n}_{t + 1}^{B} + {z}_{B}^{k}$$

where, while the transformation of the input is the same, the noise is different by walking along the two different paths because in the path B, one of the noise terms undergoes the linear transformation. This result shows also that the sequential diffusion model reduces to a diffusion model when the transformation is linear and equal to the identity transformation, which would be expected, while in the other cases, the sequential diffusion model requires the model to denoise a not-white noise, thus requiring the model to be aware to the dynamics of the system, as required.

In some instances, embodiments of the present invention can require the linear transformer to be constant.

In some examples, embodiments of the present invention can require the latent dynamic to behave such that the components are independent. This allows the capability to have a simpler dynamic and thus the computation of the noise level is able to be simplified. This requirement can be obtained by mutual independence of the features in the latent space and then using a 1×1 convolution of propagate the dynamic.

Embodiments of the present invention provide for denoising in one step or in two steps. For instance, embodiments of the present invention can either separate the denoising and the forward operator learning or have the same architecture to perform both steps. This is described above (e.g., described in FIGS. 15 and 16 above).

The algorithm for the conditional sequential diffusion model is described. For instance, the training of the Conditional Sequential Diffusion model aims at minimizing noise prediction error, which is shown below:

$${ℒ}^{CSLDM} = {𝔼}_{{ℰ}_{ϕ} ( x ) , ϵ \sim 𝒩 ( 0 , 1 ) , k} [ {\sum }_{t = 1}^{T} {‖ ϵ - {ϵ}_{θ} ( {z}_{t}^{k} , k , {τ}_{ψ} ( y ) , {x}_{< t} ) ‖}_{2}^{2} ]$$

where

$${z}_{t}^{k} = \sqrt{{\overline{a}}_{k}} {z}_{t}^{0} + \sqrt{1 - {\overline{a}}_{k}} ϵ \\ {z}_{t}^{0} = \frac{1}{σ} [ {x}_{t} - {f}_{ψ} ( {x}_{< t} ) ] \\ {\overline{a}}_{k} = {\prod }_{k = 1}^{n} ( 1 - {β}_{i} ) \\ ϵ \sim N ( 0 , I )$$

with τ_{ψ}(y) the conditioning function on the initial conditions, boundary conditions or final conditions, while ƒ_{ψ}(x_{<t}) is the prediction provided by the physical model that predicted it based either on the history or on the future ƒ_{ψ}(x_{>t}) for the reverse mode. x_{<t}, x_{>t} are the past or the future trajectories (the context).

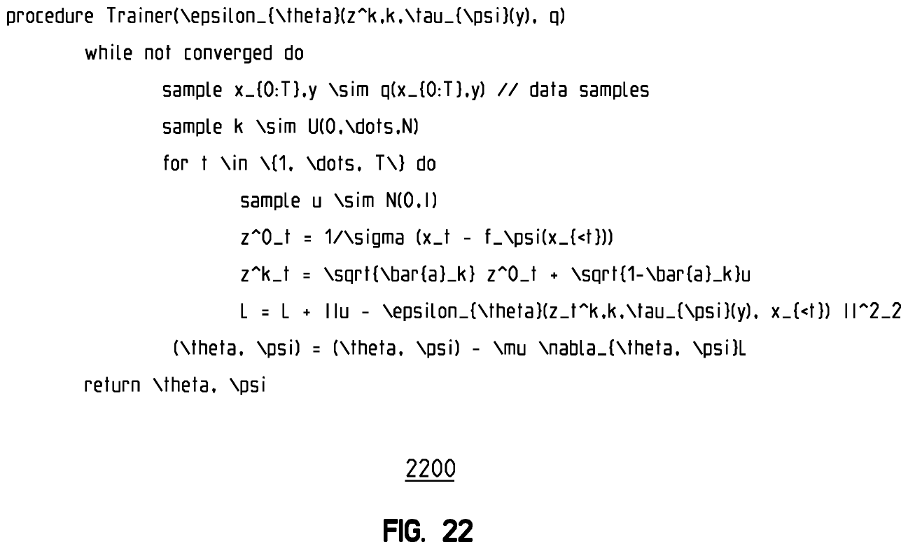

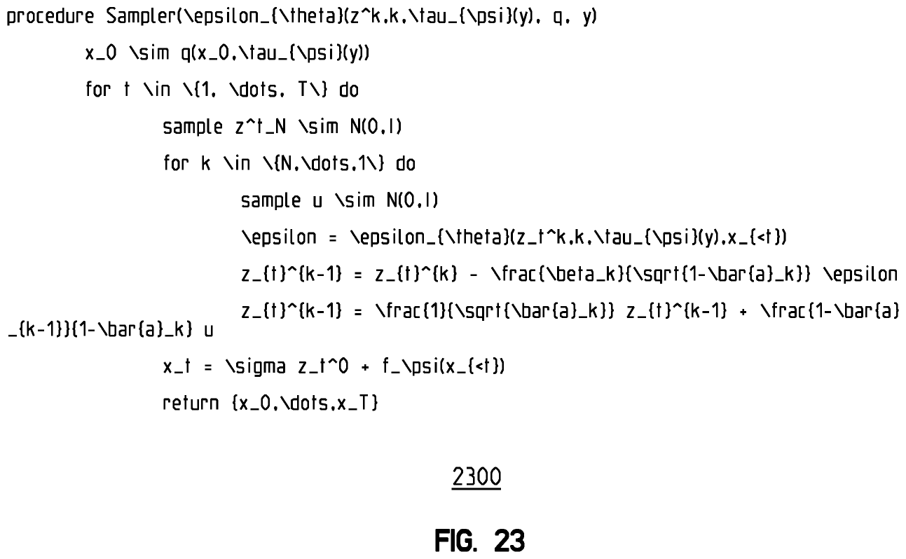

FIG. 22 illustrates exemplary code for condition sequential DM training according to an embodiment of the present invention. FIG. 23 illustrates exemplary code for sampling of the conditional sequential DM according to an embodiment of the present invention. For instance, FIG. 22 shows pseudo-code 2200 of the training phase, where for each real time t from 1 to T, embodiments of the present invention sample noise u and use, after diffusion time rescaling, to train the denoising network “\epsilon” by building the loss as the norm of the difference between the true and reconstructed noise. In this code, t is the real time and k is the diffusion time. Further, the parameters of the network “\theta” and the conditional network “\psi” are updated in a way to minimize the loss L.

FIG. 23 represents an example pseudo code 2300 of the generation phase (or sampling) where embodiments of the present invention use the trained denoising network “\epsilon” to generate a new sample. Embodiments of the present invention generate the time step t from the previous time steps <t. In this code, t is the real time and k is the diffusion time.

In some variations, embodiments of the present invention use a diffusion model having two steps: 1) diffusion process; and 2) denoising process. The diffusion model can include generative methods that include a diffusion process, which progressively distorts a data point mapping it to a noise, and a generative denoising process that approximates the reverse of the diffusion process. The diffusion process iteratively adds noise to a data to progressively transform it into the Gaussian noise. For instance, the diffusion model can use the below:

$$q ( {z}_{t} \vert {z}_{t - 1} ) = N ( {z}_{t} ; {\overline{α}}_{t} {z}_{t - 1} ; {\overline{σ}}_{t}^{2} I )$$

For αt∈R+ signal is retained and σt∈R+ added noise.

For the Markov transition model, the below is provided:

$$q ( {z}_{0} , {z}_{1} , … , {z}_{T} \vert x ) = q ( {z}_{0} \vert x ) {\prod }_{t - 1}^{T} q ( {z}_{t} \vert {z}_{t - 1} ) .$$

For the distribution of zt given x (q is normal), the below is provided:

$$q ( {z}_{t} \vert x ) = 𝒩 ( {z}_{t} \vert {α}_{t} x , {σ}_{t}^{2} I ) , \\ \overline{{a}_{t}} = {a}_{t} / {a}_{t - 1} \\ {\overline{σ}}_{t}^{2} = {σ}_{t}^{2} - {\overline{α}}_{t}^{2} {σ}_{t - 1}^{2}$$

For the reverse of the diffusion process, the below is provided:

$$q ( {z}_{t - 1} \vert x , {z}_{t} ) = 𝒩 ( {z}_{t - 1} ; {μ}_{t} ( x , {z}_{t} ) , {ς}_{t}^{2} I ) , \\ {μ}_{t} ( x , {z}_{t} ) = \frac{{\overline{α}}_{t} {σ}_{t - 1}^{2}}{{σ}_{t}^{2}} {z}_{t} + \frac{{α}_{s} {\overline{σ}}_{t}^{2}}{{σ}_{t}^{2}} x and {ς}_{t} = \frac{{\overline{σ}}_{t} {σ}_{t - 1}}{{σ}_{t}}$$

For the generative denoising process, the method learns to invert this trajectory with x unknown, and the below is provided:

$$p ( {z}_{t - 1} \vert {z}_{t} ) = q ( {z}_{t - 1} \vert \hat{x} , {z}_{t} ) ,$$

Alternatively, the prediction of the Gaussian noise is performed, and the below is provided.

$$\hat{x} = ( 1 / {α}_{t} ) {z}_{t} - ( {σ}_{t} / {α}_{t} ) {\hat{ϵ}}_{t} . \\ {\hat{ϵ}}_{t} = φ ( {z}_{t} , t )$$

Instead of the ELBO, the below is provided.

$${𝔼}_{t \sim u ( 0 , … , T )} [ T \cdot ℒ ( t ) ] \\ ℒ ( t ) = {‖ ϵ - {\hat{ϵ}}_{t} ‖}^{2}$$

Sample new data points, after training is performed. The below is provided:

$$x \sim p ( x \vert {z}_{0} ) \\ {\overline{z}}_{t - 1} \sim p ( {z}_{t - 1} \vert {z}_{t} ) \\ {z}_{T} \sim 𝒩 ( 0 , I )$$

Exemplary algorithms for the sequential diffusion model are provided below. In the below, k represents the real time and t represents the diffusion time.

```
Algorithm 1 Trainer (ϵ_{θ}(z^{k}, k, τ_{ψ}(y), q)
Require: ϵ_{θ}, τ_{ψ}, q(x_{0:T}, y), T
  while not converged do
    sample x_{0:T}, y~q(x_{0:T}, y) // data samples
    sample k~U(0, ... , N)
    for t {1, ... , T} do
      sample u~N(0, I)
      zₜ⁰ = 1/σ (xₜ − f_{ψ} (x_{<t}))
      zₜ^{k} = [symbol]  + [symbol]
      L = L + ||u - ϵ_{θ} (zₜ^{k}, k, τ_{ψ} (y), x_{<t})||₂²
    end for
    (θ, ψ) = (θ, ψ) − μ∇_{θ,ψ}L
  end while
  return θ, ψ
Algorithm 2 Sampler (ϵ_{θ} (z^{k}, k, τ_{ψ} (y), q, y)
Require: ϵ_{θ}, τ_{ψ}, T, q(x₀, y)
  x₀~q(x₀, τ_{ψ}(y)) // initial state
  for t ∈ {1, ... , T} do
    sample z_{N}^{t}~N(0, I)
  for k ∈ {N, ... ,1} do
      sample u~N(0, I)
      $\begin{matrix} ϵ = {ϵ}_{θ} ( {z}_{t}^{k} , k , {τ}_{ψ} ( y ) , {x}_{< t} ) \\ {z}_{t}^{k - 1} = {z}_{t}^{k} - \frac{{β}_{k}}{\sqrt{1 - {\overline{a}}_{k}}} ϵ \\ {z}_{t}^{k - 1} = \frac{1}{\sqrt{{\overline{a}}_{k}}} {z}_{t}^{k - 1} + \frac{1 - {\overline{a}}_{k - 1}}{1 - {\overline{a}}_{k}} u\end{matrix}$
  end for
  xₜ = σzₜ⁰ (xₜ − f_{ψ} (x_{<t})
end for
return x₀, ... , x_{T}
```

### Conditional Sequential DM Training:

```
pseudo-code
  procedure Trainer(\epsilon_{\theta}(z\hat{ }k,k,\tau_{\psi}(y), q)
    while not converged do
      sample x_{0:T},y \sim q(x_{0:T},y) // data samples
      sample k \sim U(0,\dots,N)
      for t \in \{1, \dots, T\} do
        sample u \sim N(0,I)
        z\hat{ }0_t = 1/\sigma (x_t − f_\psi(x_{<t}))
        z\hat{ }k_t = \sqrt{\bar{a}_k} z\hat{ }0_t + \sqrt{1−\bar{a}_k} u
        L = L + ||u − \epsilon_{\theta}(z_t\hat{ }k,k,\tau_{\psi}(y), x_{<t})
      ||\hat{ }2_2
    (\theta, \psi) = (\theta, \psi) − \mu \nabla_{\theta, \psi} L
  return \theta, \psi
```

### Sampling of Conditional Sequential DM:

```
pseudo-code
  procedure Sampler(\epsilon_{\theta}(z\hat{ }k,k,\tau_{\psi}(y), q, y )
    x_0 \sim q(x_0,\tau_{\psi}(y))
    for t \in \{1, \dots, T\} do
      sample z\hat{ }t_N \sim N(0,I)
      for k \in \{N,\dots,1\} do
        sample u \sim N(0,I)
        \epsilon = \epsilon_{\theta}(z_t\hat{ }k,k,\tau_{\psi}(y), x_{<t})
        z_{t}\hat{ }{k−1} = z_{t}\hat{ }{k} −
        \frac{\beta_k}{\sqrt{1−\bar{a}_k}}
      \epsilon
        z_{t}\hat{ }{k−1} = \frac{1}{\sqrt{\bar{a}_k}} z_{t}\hat{ }{k−1} +
      \frac{1−\bar{a}_{k−1}}{1−\bar{a}_k} u
    x_t = \sigma z_t\hat{ }0 + f_\psi(x_{<t})
  return {x_0,\dots,x_T}
```

While embodiments of the invention have been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiment.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

What is claimed is:

1. A method for simulating physical systems using a sequential diffusion model (SDM), wherein the SDM comprises a denoising model, comprising:
collecting training data for training the SDM;
training the denoising model using the training data such that the SDM models a forward and/or reverse problem for a simulation of a physical system over a period of time; and
generating a solution for the physical system based on training the denoising model, wherein the solution indicates a final condition of the physical system at a final instance in the period of time for the forward problem and an initial condition of the physical system at an initial instance in the period of time for the reverse problem.

2. The method of claim 1, wherein collecting the training data comprises generating simulated data, wherein generating the simulated data comprises iteratively requesting a numerical simulator to generate new data based on performance of the SDM in an active learning cycle.

3. The method of claim 1, wherein the physical system is associated with molecule generation, and wherein the training data comprises molecular data in a simplified molecular-input line-entry (SMILE) format, wherein the molecular data indicates molecules as proteins, configurations of the molecules, and atom types of the molecules, wherein the configurations of the molecules are represented in 2-D coordinates or are in 3-D space.

4. The method of claim 1, wherein training the denoising model using the training data comprises training the denoising model based on initial conditions of the physical system, boundary conditions of the physical system, observations or final conditions of the physical system, and past or future time steps of the physical system.

5. The method of claim 1, wherein training the denoising model comprises sequentially and recursively updating the SDM to predict noise or a clean input, after adding Gaussian noise to inputs of the SDM and training the denoising model to reconstruct the Gaussian noise.

6. The method of claim 1, wherein training the denoising model comprises starting with a noisy version of an input and denoising conditional to the input using the denoising model, wherein a first portion of the input is fixed and not modified and a new component of the input is generated at each diffusion step of the SDM.

7. The method of claim 6, wherein training the denoising model further comprises:
at each of the diffusion steps of the SDM, generating a denoised sequence by propagating a sequential process either in a forward direction or backward direction, wherein each propagation step obtains conditioning from a previous time step, conditioning on the input, and one or more variables to optimize upon, wherein the input is an initial condition or an observation, and wherein the one or more variables indicate boundary conditions or the initial condition.

8. The method of claim 1, wherein a condition of the SDM is on a forward model or a reverse model, wherein the forward model and the reverse model are modeled with neural networks, and wherein a boundary condition of the SDM is a discrete variable.

9. The method of claim 1, wherein the physical system is a molecular system, and wherein a condition of the SDM is an integrator based on a gradient of potential energy, wherein the gradient of the potential energy is modeled using a neural network.

10. The method of claim 1, wherein physical constraints and physical laws are used as a loss function to minimize a denoised prediction of the SDM such that the SDM is physically consistent.

11. The method of claim 10, wherein the physical system is generating a video, and wherein the physical constraints and physical laws indicate a language model and description of the video in words or sentences and/or textual description for consecutive frames of the video changes according to an externally provided distance.

12. The method of claim 1, wherein generating the solution for the physical system comprises generating a sequence of molecule configurations, wherein generating the sequence of molecule configurations comprises inputting, into the SDM, descriptions of molecules in a simplified molecular-input line-entry (SMILE) format that are converted into a 3-D format and desired properties of a generated output to determine an output, wherein the output is a 3-D description of a generated molecule in the SMILE format and indicates expected properties of the generated molecule.

13. The method of claim 1, wherein generating the solution for the physical system comprises generating a solution of a partial derivative equation (PDE) or a video, wherein the SDM is conditioned to one or more conditions, wherein the one or more conditions indicate past or future solutions, external input, observations, initial conditions, final conditions, or boundary conditions.

14. A system for simulating physical systems using a sequential diffusion model (SDM), wherein the SDM comprises a denoising model, the system comprising one or more hardware processors, which, alone or in combination, are configured to provide for execution of the following steps:
collecting training data for training the SDM;
training the denoising model using the training data such that the SDM models a forward and/or reverse problem for a simulation of a physical system over a period of time; and
generating a solution for the physical system based on training the denoising model, wherein the solution indicates a final condition of the physical system at a final instance in the period of time for the forward problem and an initial condition of the physical system at an initial instance in the period of time for the reverse problem.

15. A tangible, non-transitory computer-readable medium having instructions thereon which, upon being executed by one or more processors, alone or in combination, provide for execution of a method for simulating physical systems using a sequential diffusion model (SDM), wherein the SDM comprises a denoising model, the method comprising the following steps:
collecting training data for training the SDM;
training the denoising model using the training data such that the SDM models a forward and/or reverse problem for a simulation of a physical system over a period of time; and
generating a solution for the physical system based on training the denoising model, wherein the solution indicates a final condition of the physical system at a final instance in the period of time for the forward problem and an initial condition of the physical system at an initial instance in the period of time for the reverse problem.
